(19) United States
(12) Patent Application Publication
(10) Pub. No.: US 2024/0000396 A1
(43) Pub. Date: Jan. 4, 2024

(54) SLEEP PHYSIOLOGICAL SYSTEM AND SLEEP ALARM METHOD

(71) Applicant: Chang-An Chou, Taipei (TW)

(72) Inventor: Chang-An Chou, Taipei (TW)

(21) Appl. No.: 18/367,421

(22) Filed: Sep. 12, 2023

Related U.S. Application Data
(62) Division of application No. 17/611,134, filed on Nov. 13, 2021.

(30) Foreign Application Priority Data
May 14, 2019 (CN) 201920686690.9
Feb. 7, 2020 (CN) 202010082681.6
Feb. 7, 2020 (CN) 202010082720.2
Feb. 7, 2020 (CN) 202010082786.1
Feb. 7, 2020 (CN) 202010082863.3
Feb. 7, 2020 (CN) 202010082864.8
Feb. 7, 2020 (CN) 202010082865.2
May 8, 2020 (CN) 202010381662.3

(51) Int. Cl. A61B 5/00; A61B 5/11

(52) U.S. Cl. CPC A61B 5/746; A61B 5/1116; A61B 5/4818; A61B 5/6804; A61B 5/7282; A61B 2562/0219

(57) ABSTRACT

Sleep physiological system and sleep alarm method are disclosed. The method includes acquiring sleep respiratory information and sleep position related information of a user in a sleep duration; comparing the sleep respiratory information with a predetermined condition for deciding a sleep respiratory event; and comparing the sleep position related information with a predetermined position range; providing an alarm behavior according to a corresponding set of alarming conditions based on a comparison result between the sleep position related information and the predetermined position range so as to influence a sleep position and/or a sleep respiratory state of the user.

### CROSS-REFERENCE TO RELATED APPLICATION

This application is a divisional application of U.S. application Ser. No. 17/611,134 filed on Nov. 13, 2021 and entitled “SLEEP PHYSIOLOGICAL SYSTEM AND SLEEP ALARM METHOD”. The entire contents of the above-mentioned patent application are incorporated herein by reference for all purposes.

### FIELD OF INVENTION

The present invention is related to a sleep system and a sleep alarm method, and more particularly, to a sleep system and a sleep alarm method capable of evaluating and improving sleep breathing disorders.

### BACKGROUND OF THE DISCLOSURE

Sleep apnea is one kind of Sleep Breathing Disorders (SDB). There are three general types of sleep apnea: Obstructive Sleep Apnea (OSA), Central Sleep Apnea (CSA) and Mixed Sleep Apnea (NSA).

OSA is a sleep-related breathing disorder that involves a decrease or complete halt in airflow in the presence of breathing effort. This can lead to abrupt reductions in blood oxygen saturation (desaturation). OSA is a common sleep disorder and affects about 25^{˜}40% of the middle-aged population.

CSA results from the brain failing to signal the muscles to breathe. The neural drive to the respiratory muscles discontinues for a brief period of time. These transients may continue throughout the night for periods from ten seconds to as long as 2 to 3 minutes. CSA, similar to obstructive sleep apnea, causes a gradual asphyxiation during sleep, resulting in a brief arousal from sleep, at which time the individual's respiratory function returns to normal. Similar to obstructive sleep apnea, central sleep apnea can result in illnesses such as cardiac arrhythmias, hypertension, heart disease and/or heart failure.

MSA is a combination of obstructive sleep apnea and central sleep apnea.

Apnea Hypoxia Index (AHI) is an index of sleep apnea severity that combines the numbers of apneas and hypopneas. Combining these gives an overall sleep apnea severity score that evaluates both the number of sleep (breathing) disruptions and degree of blood oxygen saturation (blood oxygen level). The AHI is calculated by dividing the total number of apnea and hypopnea events by the number of hours of sleep. Generally, AHI values are typically categorized as 5-15/hr=mild; 15-30/hr=moderate; and >30/hr=severe.

Except for AHI, another important index for evaluating or checking sleep apnea is ODI (Oxygen Desaturation Index). The ODI is defined as the number of episodes of oxygen desaturation per hour of sleep. Typically, ODI is reported as the number of 3% desaturations (ODI3%) and/or the number of 4% desaturations (ODI4%). The difference between ODI and AHI is AHI further includes events which may cause awaken or arousal but not influence the blood oxygen level. Both ODI and AHI are correlated to sleep apnea and have validity in the diagnosis of OSA.

Further, low oxygen level is also an index for the evaluation of sleep apnea which is the ratio of the time oxygen level lower than 90% to the total monitoring time. Because AHI and ODI are both based on the happening number, it may not be able to reflect the situation that the oxygen level remains low without abrupt reductions, and the observation of low oxygen level can cover this situation. Thus, the low oxygen level is also related to sleep apnea.

Most patients with OSA have more OSA events when in a supine sleep position. This is because when in a supine sleep position, the shape and size of the upper airway are more easily altered owing to gravity so as to increase the likelihood of obstructing the airway. With positional obstructive sleep apnea, the AHI in supine position is often twice as high as opposed to other sleep positions. It is thought that about 70%^{˜}80% of people with positional obstructive sleep apnea have mild to moderate OSA symptoms, in which up to 87% of Asia patients with mild OSA can be classified as patients with positional obstructive sleep apnea.

Another common sleep-related breathing disorder is snoring which affects about 20%^{˜}40% of the population. Snoring is the hoarse or harsh sound that occurs when air flows past relaxed tissues in the throat, causing the tissues to vibrate as breathing. Snoring is the most common symptom that accompanies OSA and is regarded as the precursor before OSA. Since snoring is also caused by the narrowed upper airway, the sleep position also influences the severity of snoring.

Generally, when the upper airway starts to collapse, the snoring related to sleep position happens first. As it becomes severe, snoring happens even in a non-supine position, and then, the symptom becomes to be mild OSA and the correlation between the snore and the sleep position is gradually reduced. Further, with the severity of OSA increases, the correlation between OSA and sleep position is also reduced accordingly.

Sleep positional training (SPT) is a procedure to treat positional OSA and position snoring. Recently, the newly developed SPT device is implemented to mount the position sensor, e.g, the accelerometer, around the longitudinal axis of the human body, e.g., neck, chest and abdomen, for detecting the user's sleep position, and provides vibration alarms as the user is in a supine position, so as to prompt the user to change to a non-supine position. This is a simple but effective method.

However, this kind of training still can be improved. For example, since the severity of OSA or snoring of every patient is different, if an evaluation before training can be executed, the training program may target different patients and the information about training also can be provided. Further, during SPT, if the information about sleep and respiration which can be used to adjust the setting parameters of the device can be provided, the effect of training can be further improved.

In addition, except for sleep positional training, if other training methods, e.g., for sleep disorders not related to sleep position or for further enhancing on the basis of SPT, also can be provided, it will be even more helpful.

### SUMMARY OF THE DISCLOSURE

An object of the present invention is to provide a sleep physiological system which adopts a dispersed deployment framework, so that when evaluating sleep disorders and performing a sleep position training and/or a sleep physiological feedback training, depending on different demands, the user can select to use an appropriate physiological sensor for acquiring appropriate sleep physiological information, and also can select the type and mounting position for alarm provision, which facilitates a more accurate reflection of the sleep physiological condition and an improvement of training effects.

Another object of the present disclosure is to provide a sleep physiological system which can be mounted on different body portions of a user through combining with at least a wearable structure and also includes one or more physiological sensor for acquiring different physiological information as being mounted on different body portions, thereby this single system being able to provide multiple functions depending on different timings and purposes.

Another object of the present disclosure is to provide a sleep physiological system which can acquire multiple sleep physiological information at one single position through selecting the type of physiological sensor, the wearable structure and/or the mounting position of the body, so that the evaluation of sleep disorder can be more accurate and the training effect also can be improved.

Another object of the present disclosure is to provide a sleep physiological system which adopts an oral closing auxiliary to affect the upper airway for improving sleep disorder and at the same time employs the physiological sensor to acquire sleep respiratory information for revealing the improvement.

Another object of the present disclosure is to provide a sleep physiological system which is positioned between the nose and the mouth through a wearable structure for employing an airflow sensor to acquire breathing flow variations and also employing another physiological sensor to acquire sleep physiological information and/or sleep respiratory events during sleep.

Another object of the present disclosure is to provide a sleep physiological system and a sleep alarm method. In the sleep alarm method, a sleep physiological system is utilized to acquire a sleep position related information and at least a sleep respiratory information of a user, different sets of alarming conditions are provided according to a comparison result between the sleep position related information and a predetermined position range, so as to decide a corresponding alarm behavior, and according to the alarm behavior, at least an alarm is produced and provided to the user for achieving the effects of influencing the user's sleep position and/or the user's breathing condition during sleep.

### BRIEF DESCRIPTION OF THE DRAWING FIGURES

A more complete understanding of the embodiments of the present disclosure may be derived by referring to the detailed description and claims when considered in connection with the following illustrative figures.

FIG. 1 is a block diagram illustrating a sleep physiological system according to the present disclosure.

FIG. 2 is a schematic view illustrating possible positions for placing physiological sensors according to the present disclosure.

FIG. 3 is a flow chart illustrating a process for improving sleep apnea/hypopnea according to the present disclosure.

FIG. 4 is a flow chart illustrating a process for evaluating the relationship between sleep positions and snoring according to the present disclosure

FIG. 5 is a flow chart illustrating a process for evaluating the relationship between sleep positions and sleep apnea/hypopnea according to the present disclosure.

FIG. 6 shows PPG signal and the time domain features.

FIG. 7 is a flow chart illustrating how to perform a sleep position training and/or a sleep respiratory feedback training in a sleep duration according to the present disclosure.

FIGS. 8A-8C illustrate exemplary embodiments of an adhesive wearable structure with electrodes according to the present disclosure.

FIGS. 9A-9C illustrate exemplary embodiments of an ear plug type wearable structure according to the present disclosure.

FIG. 10 is schematic view illustrating an airflow sensor mounted between the nose and the mouth according to the present disclosure;

FIG. 11 is a schematic view illustrating the possibility of a housing for combining with different wearable structure according to the present disclosure.

FIGS. 12A-12B are schematic views illustrating exemplary embodiments of an oral closing auxiliary according to the present disclosure.

FIGS. 12C-12E are schematic views illustrating exemplary embodiments of combinations between a chin belt and a head-mount structure according to the present disclosure.

### DETAILED DESCRIPTION OF EXEMPLARY EMBODIMENTS

The description of exemplary embodiments of methods, structures, devices and systems provided below is merely exemplary and is intended for purposes of illustration only; the following description is not intended to limit the scope of the disclosure or the claims. Moreover, recitation of multiple embodiments having stated features is not intended to exclude other embodiments having additional features or other embodiments incorporating different combinations of the stated features. For example, various embodiments are set forth as exemplary embodiments and may be recited in the dependent claims. Unless otherwise noted, the exemplary embodiments or components thereof may be combined or may be applied separate from each other.

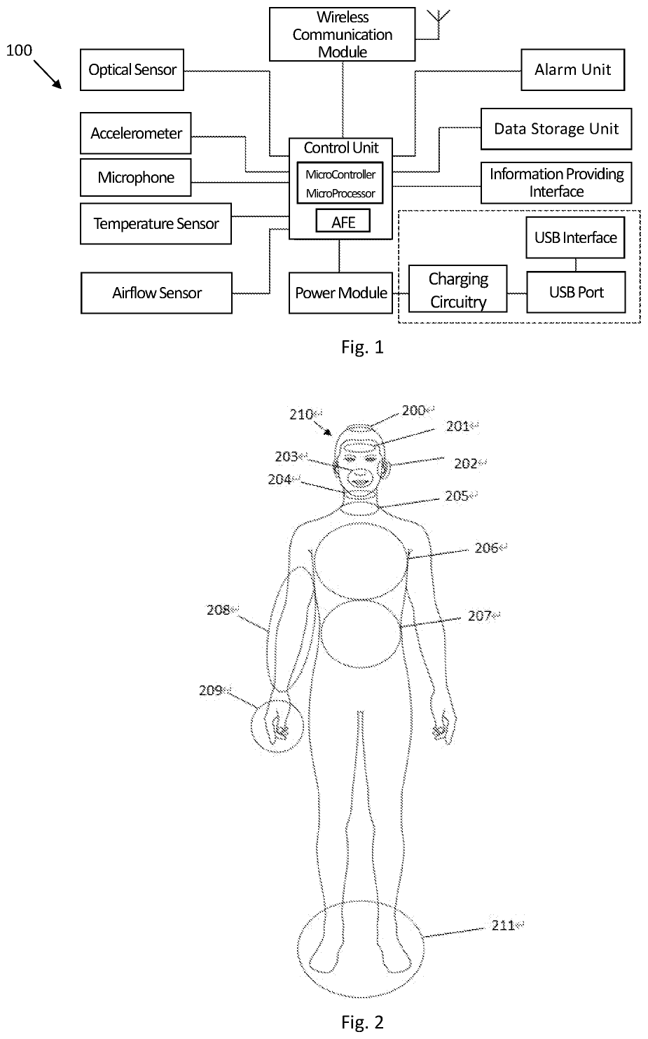

FIG. 1 is an illustration of a possible block diagram of the system. All the components are connected to the control unit. The control unit is in particular contains at least one microcontroller/microprocessor with a preloaded program to handle the communication between and the control of the hardware components. The control unit makes it possible to transfer all the signals between the different hardware components and external applications/products connected to the device/system. Furthermore, it enables the programming of the behavior of the device/system and so to tell it how to respond to different operation situations.

The control unit may include an analog front end (AFE) circuitry for processing acquired physiological signals, such as analog-to-digital conversion, amplifying, filtering and other processes.

The system may include an optical sensor, which includes at least a photo emitter, such as LED, and at least a photodetector, such as photodiode, for obtaining a photoplethysmography (PPG) signal. Light is emitting into the tissue and the light reflected by or penetrating through blood in the blood vessel is measured by the photodetector. Thus, all physiological information that can be derived from the PPG signal is called blood physiological related information in the present application. The PPG signal consists of the fast moving component (“AC component”) reflective of pulse waves sent through the arteries by contracting heart muscle, and the slow moving component (“DC component”) reflective of slower changes in tissue blood volume, which is caused by respiratory effort, the activity of autonomic nervous system (ANS system) and Mayer waves. Through analyzing PPG signals, physiological information related to vascular stiffness and blood pressure also can be obtained.

Generally, in accordance with the type and number of photo emitter and photodetector contained in the photo sensor, the derived physiological information can be different. For example, the photo sensor may include at least a photo emitter, such as LED or LEDs, Green/IR/Red/Blue or White, which is composed of many colors, preferred, and at least a Photodetector to get PPG waveform for pulse rate/heart rate and other blood physiological related information, such as respiratory information. When measuring pulse rate/heart rate, green light and visible light with shorter wavelength are the currently used light source, and it is focused on interpreting the AC component. When a person breathes, the pressure inside the chest cavity, called the intra-thoracic pressure, changes with each breath. As a person inhales, the chest expands resulting in a decrease in intra-thoracic pressure, which draws air into the lungs. During an exhalation, the intra-thoracic pressure increases and forces air out of the lungs. These changes in intra-thoracic pressure also cause changes in the amount of blood returned to the heart via veins and the amount of blood pumped by the heart into arteries. This effect on the peripheral blood volume can be estimated by detecting a temporary increase in the DC component. In the present invention, breathing related information derived from analyzing PPG waveform is called low frequency respiratory behavior. Further, because heart rate is controlled by the ANS system, breathing that influences the ANS system may also cause a variation in heart rate, which is the so-called respiratory sinus arrhythmia (RSA). During an inhalation, heart rate will increase and during an exhalation, heart rate will decrease. The respiration can be derived by observing heart rate variation. It is called RSA respiratory behavior in the present application. Further, the respiration related physiological information acquired by the optical sensor is collectively called respiratory behavior.

The photo sensor also may include at least two photo emitters, such as LEDs, IR/Red/Green preferred, and at least a Photodetector to get the blood oxygen saturation (SPO2) data, pulse rate/heart rate, and other blood physiological related information, such as respiratory information. When measuring SPO2, two photo emitters emit lights in two different wavelengths and the at least a photodetector measure reflection or absorption of light wavelengths typically reflected or absorbed by oxygenated hemoglobin (HbO2) and desoxygenated hemoglobin (Hb). The results are compared to determine the oxyhemoglobin concentration. Thus, the position for measuring SPO2 should be preferably where the light can be emitted in the artery, such as fingers, palm, toes and sole, for example, toes/sole are usually employed to measure SPO2 of an infant. The two different wavelengths can be, for example, IR and Red, or Greens in two wavelengths, such as Greens in 560 nm and 577 nm, without limitation.

The wavelength ranges of light sources may be about 620 nm to 750 nm in Red, about longer than 750 nm in IR and about 495 nm to 580 nm in Green. When in use, the usually used are, for example, Red in 660 nm, IR in 895 nm, 880 nm, 905 nm or 940 nm, and Green in 510-560 nm or 577 nm. Based on different purpose, other wavelengths also can be used, for example, when heart rate is the only target, Blue and White which is composed of many colors also can be used. Therefore, for accuracy, the term “wavelength combination” is used instead of “wavelength” hereinafter.

Particularly, the photo sensor may also contain three wavelength combinations. In one embodiment, a first light source is implemented as IR with a first wavelength combination, a second light source is implemented as Red with a second wavelength combination, and a third light source is implemented as Green, Blue or White with a third wavelength combination. Red and IR are used to acquire SPO2 data, and Green, Blue or White is used to acquire pulse rate/heart rate. In another embodiment, a first light source is implemented as IR or Red with a first wavelength combination, and a second and a third light sources are implemented as Green, Blue and/or White with a second and a third wavelength combinations. Among three wavelength combinations, two are used to acquire SPO2 data and the other is used to acquire pulse rate/heart rate. In another embodiment, three light sources are all implemented as Green respectively with a first, a second and a third wavelength combinations. Two wavelength combinations are used to acquire SPO2 data and the other is used to acquire pulse rate/heart rate. Because different portions of human body can acquire different kinds of blood physiological related information, by having light source capable of emitting multiple wavelength combinations, it will be helpful that only one device also can be moved to different body portions for obtaining different blood physiological related information.

When there are three light sources, the number and position of photodetector accordingly can be adjusted. For example, it can be implemented as two photodetectors, in which one cooperates with one IR and one Red light sources to acquire SPO2 data, and the other cooperates with two Green light sources to acquire pulse rate/heart rate. Alternatively, only one photodetector can cooperate with one IR, one Red and one Green light sources to acquire SPO2 data and pulse rate/heart rate. Alternatively, only one photodetector also can cooperate with one IR and one Red light sources to acquire SPO2 data and cooperate with three Green light sources to acquire pulse rate/heart rate. There is no limitation.

For receiving IR/Red light, it will be better to employ a smaller size of photodetector so as to avoid the saturation caused by other environmental lights. For receiving Green, Blue or White light, the size of the photodetector can be bigger so as to acquire more effective reflection lights. Further, a blocking process is also useful, for example, by adapting a filtering material for isolating low frequency IR and get signals with better S/N ratio.

As acquiring heart rate, for eliminating noises, such as environmental noises, noises caused from body movements, more than two light sources (without limiting the wavelength combinations, such as two Greens or others) can be used, and through the digital signal process, e.g., adaptive filter, or mutual subtraction of acquired PPG signals, the noises can be cancelled.

The system further may include a posture sensor, usually a gravity sensor, preferably a three-axes (MEMS) accelerometer, to define the posture of the device in three dimensions that is directly related to the posture of the body of the user. The accelerometer returns values for the accelerations measured in all three dimensions x, y and z. From these values, not only sleep posture, other sleep information also can be derived, such as actigraph, movement, upright/laydown positions. And, by analyzing the actigraph during sleep, the information of sleep state/stage can be derived. Other kinds of sensor also can be used, e.g., gyroscope, magnetometer.

The system may include a microphone. The microphone returns values for the frequency and amplitude of the measured sound. By using an audio transducer, it may detect sounds during the sleep period, e.g., snoring or other respiratory activity, with appropriate filter designs.

The system may include a snore detector. The snore detector can be a microphone which detects the sound of snoring. The snore detector also can be an accelerometer or piezoelectric vibration sensor which detects the vibrations of the body cavity. The vibration caused by snoring may be detected on several body positions, such as torso, neck, head and ears. Torso and head are preferred; especially nasal cavity, throat and chest are good at transmitting vibrations caused by snoring. Compared to detecting sounds, the detection of vibration won't be influenced by environmental noises or covering on the body, such as quilts. Preferably, the accelerometer used as the posture sensor is also implemented to detect snoring. The snore information, such as strength/duration/counts can be obtained by applying appropriate filter designs and other well-known techniques to the original vibration signals.

The system may also include a temperature sensor to detect device temperature, environment temperature, or body temperature to provide further physiological information of the user during the sleep.

The system may also include an airflow sensor, e.g., thermistors, thermocouples or a nasal cannula/pressure transducer, arranged between nose and mouth for detecting the variation of breathing flow. Thermistor and thermocouple can be selected to have two detecting points near nostrils or three detecting points near nostrils and mouth.

The system may also include an accelerometer. The accelerometer can be positioned on the torso for detecting movements of chest and/or abdomen during inhalation and exhalation. The accelerometer also can be used to detect pulsations of vessels so as to obtain heart rate. Since vessels are distributed through the whole body, there is no limitation to the position for acquiring heart rate, for example, head, chest and limb are all preferable.

The system may include at least two electrodes for detecting body resistance by being arranged on the torso, such as the chest or the abdomen. The resistance is generated by the movements of chest and/or abdomen during breathing, so that through analyzing the resistance variations, the information related to respiration can be revealed, such as respiratory effort, respiratory amplitude and respiratory frequency.

The system may include a piezoelectric motion sensor being arranged on the torso to measure displacement variations caused by the volume changes of the chest or abdomen during respiratory cycles. It can be implemented as belt(s) or patch(s).

The system may include a RIP (Respiratory Inductance Plethysmography) sensor arranged on torso to measure the volume change of chest and/or abdomen during respiration. It is usually implemented as a wearable chest or abdominal belt.

The system may include at least two ECG electrodes arranged on the torso and/or limb(s). By analyzing electrocardiograms, more detailed heart activities can be revealed, e.g., to obtain an accurate heart rate, to know if there is arrhythmia, and to calculate HRV (Heart Rate Variability) which is related to ANS activities. All the information can help for understanding sleep state and status.

The system may include at least two EEG electrodes, at least two EOG electrodes and/or EMG electrodes. EEG electrodes can be arranged on head and/or ear to obtain EEG signals, EOG electrodes can be arranged near eyes or forehead to obtain EOG signals and EMG electrodes can be arranged on the body to obtain EMG signals. By analyzing EEG signals, EOG signals and/or EMG signals, it will be able to obtain information related to sleep quality, such as sleep state/stage, sleep cycle.

When acquiring electrical physiological signals, it often employs signal acquiring electrode with DRL (Driven Right-Leg) electrode, wherein the signal acquiring electrode is used to acquiring electrical physiological signals and DRL electrode is used to eliminate common mode noises, such as 50 Hz/60 Hz power noises, and/or to provide the body potential level for matching a level potential. In practice, depending on real situation, the arrangement of electrodes can be flexible, for example, two signal acquiring electrodes can be employed to adapt two-electrode mode for acquiring electrical physiological signals, or an additional DRL electrode can be further employed to adapt three-electrode mode.

Generally, there are two types of electrodes, wet electrodes and dry electrodes. Wet electrodes need to employ a conducting medium for achieving the contact with skin, e.g., conductive gel, conductive paste or conductive liquid. The most used wet electrodes are cup electrodes with conductive paste and electrode patches with preformed conductive gel. On the other hand, dry electrodes do not need to employ conductive medium and can acquire electrical physiological signals through directly contacting the skin or by being implemented as non-contact electrodes, such as capacitive, inductive or electromagnetic type electrodes. Dry electrodes can be made of many kinds of materials only if the material is capable of sensing the electrical potential of human body, e.g., metal, conductive fiber, and conductive silicone. Usually, the electrode on the surface of a device is implemented as dry electrode for simplifying the operation process.

The heart rate also can be used to get information related to sleep states/stages. Because heart rate varies with sleep stage changes, e.g., deep sleep stage and non-deep sleep stage have different heart rate variations, sleep stages can be revealed by observing heart rate variations during sleep. Further, other analysis methods for heart rate also can be used to get the information related to sleep stages. For example, HRV analysis can be used to know ANS activities which are relative to the change of sleep stages, and HHT (Hilbert-Huang transform) and other suitable methods also can be used to analyze heart rate. Usually, heart rate and body movement are observed at the same time to decide the information related to sleep stages.

The system may also include an alarm unit. Many types of alarm are possible including: audible, visual, tactile, e.g., sound, electronic stimulation, vibrotactile, or any other may be applied to notify the user. The use of vibrotactile feedback, such as a vibration motor, is preferred because it is comfortable and does not disturb the sleep rhythm of the user or partner of the user. However, in some circumstances, the alarm unit may include a speaker or earphones for audible feedback, or LEDs for visual feedback.

The system may include an information providing interface, preferably a LCD or LED display to transfer information to the user to indicate, e.g., physiological information, statistic information, analysis results, stored events, operation mode, alarm content, progress, battery status, and more.

The system may include a data storage unit, preferably a memory, such as an internal flash memory or a removable memory disk, to store detected and measured physiological information.

The system may include a communication module which can be a wireless module, such as a Bluetooth, BLE, Zigbee, WiFi, RF or other communication protocol, and/or a USB interface to communicate with external devices, may include but not limit to, a smartphone, a tablet computer, a notebook computer, a personal computer, or a smart watch, a smart band, and other wearable devices. The communication enables the exchange of information between those devices and enables options for information feedback, remote control and monitoring.

The system may include a power module, such as a coin cell, alkaline battery, or rechargeable Li ion battery. The system may have charging circuitry, such as inductive charging circuitry, or charged by the USB port or spring pins optionally.

Please refer to FIG. 2 which illustrates the positions capable of placing the above physiological sensors and alarm unit during sleep. The sleep physiological signals that can be acquired and the sensor(s) related thereto are described below.

Sleep position can be acquired by placing the position sensor around the longitudinal axis of human body, including a region of the top of head 200, a region of forehead 201, a region of ear 202, a region near nose and mouth 203, a region of chin 204, a region of neck 205, a region of chest 206 and a region of abdomen 207. And, both the front surface and the back surface of human body can be used to place the position sensor without limitation. The most representative regions are the torso and the neck.

Blood oxygen saturation can be acquired by placing the optical sensor at the region of forehead 201, the region of ear 202, the region near nose and mouth 203, a region of arm 208, a region of fingers 209 and a region of feet 211.

Heart rate can be measured by the optical sensor almost at any position of human body. The most used positions are the region of fingers 209, the region of arm 208, the region of ears 202 and a region of head 210. Further, an accelerometer with high sensitivity also can be used to detect the vibration of blood vessel caused by blood pulses so as to obtain the heart rate. There is also no limitation to the detecting position of the accelerometer, such as the head, the chest and the limbs are all workable positions.

Respiratory effort is the activity of chest and abdomen caused by respiration and can be measured by the accelerometer, the piezoelectric motion sensor, the RIP sensor or the electrodes for detecting body resistance at the chest region 206 and/or the abdomen region 207.

Respiratory behavior, as described above, is the collection of the respiration related physiological information acquired by the optical sensor, including the low frequency respiratory behavior obtained by analyzing the PPG waveform and the RSA respiratory behavior obtained by calculating the heart rate. Therefore, the position for acquiring respiratory behavior has no limit. The most used positions are the region of fingers 209, the region of arm 208, the region of ears 202 and the region of head 210.

Breathing flow variations can be measured by the flow sensor, such as thermistors, thermocouples and the nasal cannula/pressure transducer, at the region near the nose and mouth 203.

Snoring related information (sounds of snoring) and sounds of breathing can be detected by the microphone at any position even not on the body, e.g., detected by the microphone of a cell phone.

Snoring related information (vibrations of body cavity) can be measured by the accelerometer or the piezoelectric vibration sensor at the region of head 210, the region of neck 205, the region of chest 206 and the region of abdomen 207.

EEG (Electroencephalography) signals can be acquired by EEG electrodes at the region of head 210.

EOG (Electrooculography) signals can be acquired by EOG electrodes at the region of forehead 201.

EMG (Electromyography) signals can be acquired by EMG electrodes with position limit, such as the region of forehead 201 and the region of chin 204.

Actigraph can be acquired by the accelerometer at any desired position.

Sleep stages can be acquired by the optical sensor and/or the accelerometer at any desired position, or by EEG electrodes, EOG electrodes and/or EMG electrodes at the region of head. Further, through analyzing the sleep stages, e.g., the ratios of deep sleep and non-deep sleep of the whole sleep duration, the sleep quality can be revealed.

Furthermore, the alarm unit for providing tactile alarms can be placed at any position of body surface capable of sensing the vibrations. The alarm unit for providing audible alarms is preferably positioned near the ear, for example, when air conduction audible alarms are employed, it will be better to place near the ear canal, and when bone conduction audible alarms are employed, more positions, such as the skull, preferably no hair region, and the region near the ears, can be used to place the alarm unit. More than one type of alarms can be provided, for example, vibrations and sounds can be provided at the same time. Even only one type of alarm is employed, it also can have variety, for example, the tactile alarms can have different combinations according to the strength, frequency and/or duration thereof, which not only provide selectivity for user, but also keep the body feeling the vibrations.

Noted that the region of ear 202 includes the front side and the back side of auricle, the ear canal, the head portion near the ear, the region of arm 208 includes the upper arm, the forearm and the wrist, and the region of neck 205 includes the front side and the back side of the neck.

Further, various kinds of wearable structures can be utilized to install the sensor, e.g., a housing having the sensor mounted therein. For example, a belt can be used to surround the head, the arm, the finger, the neck and the torso. An adhesive structure can be adhered to the body surface, such as the forehead, the torso. A magnetic or mechanical clamp can be used to clamp a portion of the body, such as the finger and the ear, or to clamp an object located on the body surface, such as the clothes and the belt surrounding the body. A hanging element can be hung on a portion of the body, such as the auricle.

As disclosed above, the same kind of physiological information can be acquired by different kinds of physiological sensors and at different body regions. During sleep, more than one kind of physiological sensors can be used, more than one kind of physiological signals can be acquired and/or more than one body positions can be used to place the sensors. In practice, it is possible to combine all possibilities for various kinds of needs. Therefore, the following embodiments are only for illustration and not for limitation.

The PPG signals acquired by the optical sensor, except for being used to acquire blood oxygen saturation for calculating ODI values and low oxygen level, also will have other variations which can be used to determine if there is any apnea/hypopnea happened during sleep.

OSA causes bradycardia and an increase of PWA (Pulse Wave Amplitude). After the obstruction of breathing ends, the heart rate increases and the blood vessel constricts, this is called a heart rate variation sleep event in the present disclosure. Further, it is also reported that sleep respiratory events and arousals will cause more variations in PWA and/or PA (Pulse Area) compared to in HR (Heart Rate) and/or PPI

### (Peak-to-Peak Interval)

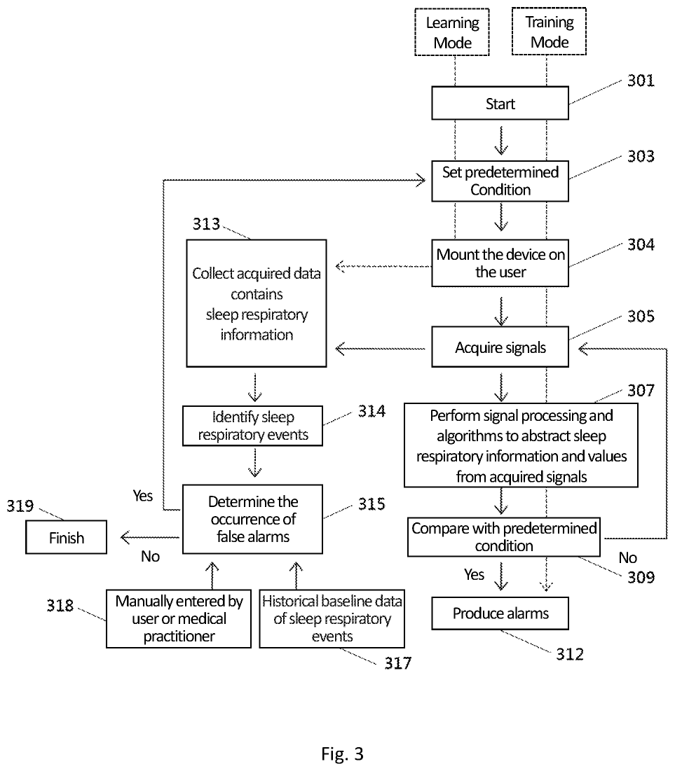

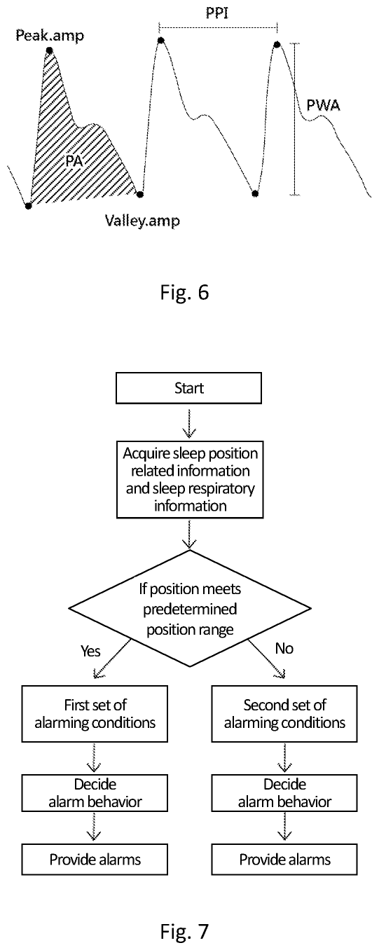

As shown in FIG. 6, the PPI is defined as the time difference between two consecutive peaks of the PPG signal. At first, the peak of each cycle of the PPG signal (peakAmp) was detected and the time stamps of all peakAmp points were stored in a vector. The PPI was calculated as the time difference between consecutive peakAmp points (see FIG. 3). To obtain accurate results, a reasonable range could be set for the PPI value, for example, PPI<0.5 s (>120 beats/min) or PPI>1.5 s (<40 beats/min) was considered ectopic and removed.

The PWA is the difference between the peak amplitude (Peak.amp) and valley amplitude (Valley.amp). The peakAmp and valleyAmp are the maximum and minimum amplitude points of each PPG cycle. At first, all peakAmps and valleyAmps were detected as the local maximum and minimum points of the PPG signal. In the case of missing peakAmp points, the next valleyAmp point was also discarded. Finally, PWA was calculated by subtracting valleyAmp from the immediately preceding peakAmp. Since peakAmp and valleyAmp points were only detected in pairs and otherwise discarded, there was no error in the PWA value introduced due to one of them missing. In addition, if there were any ectopic Peak.amp points, they were discarded by the filtering process mentioned in PPI feature extraction.

The feature PA represents the area of the triangle that consists of one Peak.amp point and two Valley.amp points (Refer to FIG. 6). Similar to PWA feature extraction, all Peak.amp and Valley.amp points were detected as the local maximum and local minimum points in the PPG signal. And since the time stamp (i.e., sample number of each point) was also recorded, the Pulse Area can be calculated from each pulse waveform.

RIIV (Respiratory-induced intensity variations) signals, that is caused by respiratory synchronous blood volume variations, can be extracted from the PPG signal by filtered with a bandpass filter (e.g. 0.13-0.48 Hz, 16th degree Bessel filter), which suppressed the cardiac-related variations and the frequencies below the respiratory frequency in the PPG signal, such as reflective changes in sympathetic tone and reflect efferent vagal activity.

Thus, for detecting sleep apnea/hypopnea and its onset, the above described sleep respiratory events, namely, PPI, PWA, PA which are extracted from PPG waveform and RIIV which is obtained by the optical sensor, also can be used as indications.

Accordingly, the present disclosure defines:

Sleep physiological information at least includes sleep position related information, sleep stage, sleep actigraph, blood oxygen saturation, heart rate, respiratory effort, respiratory frequency, respiratory amplitude, breathing flow variations, respiratory behaviors, variations of breathing sounds, snoring related information, ECG signals, EEG signals, EOG signals, and EMG signals.

Sleep respiratory information at least includes blood oxygen saturation, heart rate, respiratory effort, respiratory frequency, respiratory amplitude, breathing flow variations, respiratory behaviors, variations of breathing sounds, and snoring related information.

Sleep respiratory events include blood physiological related sleep respiratory events (ODI event, low oxygen level event, heart rate variation sleep respiratory event), snore event, apnea event and hypopnea event.

Following, the present disclosure provides a sleep respiratory feedback training based on the sleep respiratory event(s), and FIG. 3 illustrates the flow chart of utilizing the sleep respiratory feedback training to improve sleep breathing disorders.

The process is monitored by a program, in which when the sleep respiratory information meets a predetermined condition during sleep, the alarm unit is triggered to generate alarms, such as audible, visual and/or tactile alarms, so as to introduce awakeness or arousal which is sufficient to interrupt the sleep respiratory event(s) to the user for achieving the effect of stop sleep apnea/hypopnea. If there is no arousal detected, the strength of alarm will be increased as the next sleep respiratory event happens according to the sleep respiratory information.

This method of monitoring the sleep respiratory event and the onset thereof and briefly arousing the patient from sleep on a regular and continuous basis is a form of biofeedback that is used to prevent sleep apnea/hypopnea. Upon being subjected to repeated sleep apnea/hypopnea while using the system of the present disclosure, the patient reflexively learns to take several deep breaths when an event occurs, and to return to sleep. According to the research and experiment, this conditioned response to the alarm decreases or eliminates sleep apnea/hypopnea effectively over a period of usage time.

The predetermined condition can be changed with the acquired sleep respiratory information, e.g., a predetermined SPO2 level, a predetermined heart rate variation. Further, it is preferable that the alarm condition is initially programmed, and then subsequently adjusted for each user. The dynamic adjustment of the thresholds serves to reduce the incidence of false alarms, and to improve the accuracy of sleep respiratory event detection.

In one embodiment, the software program may reside within a wearable device that acquires sleep respiratory information. In another embodiment, the software program may reside in an external device, such as a smart device, e.g., a smartphone, a smart watch, a smart ring, a smart glasses, or a smart earphones, a tablet, a notebook, or a computer.

The flow starts at step 301, and then predetermined conditions are set step 303. The predetermined conditions are values at which an alarm is activated. In some embodiments, the predetermined conditions may be set within the software program 300 automatically or by using default values. Alternatively, these values may also be determined and entered manually by the user or a medical practitioner, as in Step 318, and may be changed based on patient specific information. The predetermined conditions are set for threshold conditions/values may include but not limited to, such as the blood oxygen level, the heart rate, ODI, or PA.

In the learning mode, the software program 300 begins to acquire physiological signals at step 305. Sleep respiratory information is collected with a wearable physiological device and transferred to the software program 300 using data transfer techniques known to those skilled in the art. The software program 300 also collects acquired data that contains sleep respiratory information at step 313. The acquired data is stored in a memory or a database using techniques known to those skilled in the art. Then, the sleep respiratory event is identified at step 314, e.g., by analyzing the collected sleep respiratory information.

At step 305, the software program 300 will compare the acquired data to historical baseline data of sleep respiratory event 317. The historical baseline data 317 may, in some embodiments, contain respiratory information such as heart rate values and blood oxygen content values that are provided through the guidance of a medical professional. The historical baseline data 317 may provide PPG waveforms, heart rate values, blood oxygen values, and other medical data that indicates the onset of sleep respiratory event in a user. In some embodiments, the historical baseline data 317 may be obtained from the historical readings of the user, from popular sources of historical baseline data of sleep respiratory event, such as MIT-BIH Polysomnographic Database, or from statistically derived data. In step 315, the acquired data is compared to historical baseline data 317 to determine the occurrence of false alarms during a specified time period. If false alarms are found, adjustments are made to predetermined conditions in step 315 to ensure that a sleep respiratory event is properly detected. If no false alarms are detected, or a small number of false alarms are detected that are within an acceptable pre-defined range either within the software program 300 or the user, there will be no adjustments made to the predetermined conditions in step 315, and goes to Finish status 319.

In the training mode, return to step 305, the software program 300 begins to acquire physiological signals in this step, and then in step 307 to perform signal processing and correspondent algorithms to abstract sleep respiratory information and related values from the acquired signals/data. After step 307, the software program 300 is continually checking in step 309 to determine if the predetermined conditions are matched by comparing the results obtained in step 307 with the predetermined conditions set in step 303. If the predetermined conditions have not been matched in step 309, sampling continues with no further processes started. In step 309, if a predetermined condition is matched, an alarm behavior is determined that activates the production of an alarm 312. The alarm will cause the patient to briefly awaken, take several deep breaths and return to sleep, thus ceasing the apnea/hypopnea condition. This process of monitoring, alarming (and adjusting predetermined conditions) continues throughout the training mode. The result of said process is a gradual reduction in the frequency and quantity of apnea/hypopnea events.

The learning mode and training mode may be switched dynamically, either automatically or set by the user manually, that can be executed in the same night or separate nights to optimize the treatment effectiveness, without limitations.

Following, the present disclosure provides the process to evaluate and improve a positional sleep disorder.

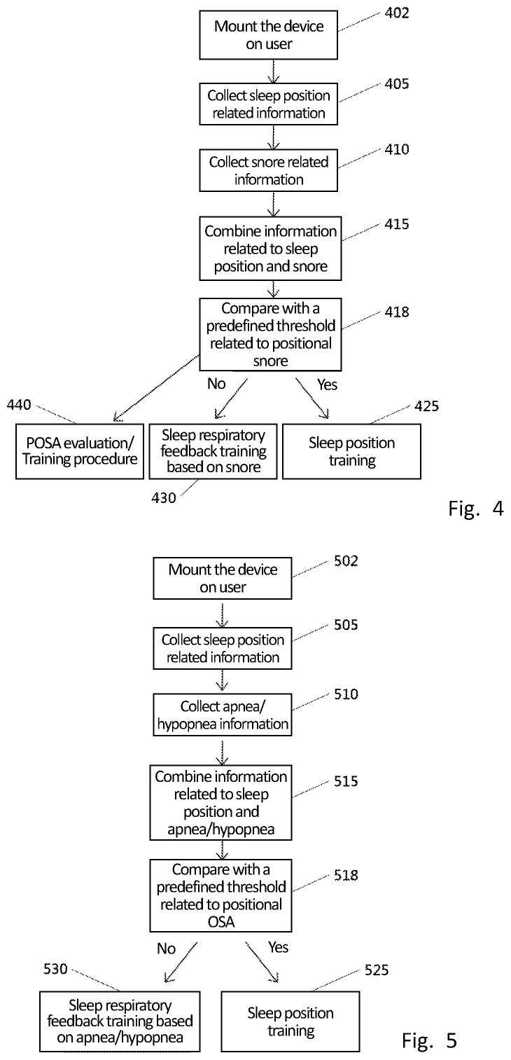

Please refer to FIG. 4 which is a flow chart illustrating the steps to evaluate the relation between the sleep positions and the snoring and provide a corresponding prevention method. At step 402, the device is mounted on a user through a wearable structure.

At step 405, once the wearable device is mounted, the controller unit initiates data collection to acquire the sleep position related information while the user is asleep. The collected data can be transmitted to an external device via the wireless communication module, or can be saved into a memory in the wearable device first, and then transmitted to an external device for later analysis. Now referring to 410, the Snoring related information is collected, the possible sensors include, but not limited to, a microphone, a piezoelectric vibration sensor, an accelerometer, either implemented on a wearable device or an external device, such as smartphone, without limitation.

Now at step 415, both the sleep position related information and the snoring related information are combined, so that a correlation may be calculated by a software program. For example, the supine snore index is defined as the number of snore events per hour while lying in supine position, the non-supine snore index is defined as the number of snore events per hour while lying in supine position, and the snore index=supine snore index+non-supine snore index. A supine-dependent snorer is defined as having a supine snore index higher than their total non-supine snore index. At step 418, a pre-defined threshold is compared with, for example, the ratio of supine snore index and non-supine snore index, or other comparisons are possible. If the threshold is exceeded, the user is identified as a positional snorer, and then may take a SPT (sleep position training) at step 425. Otherwise, the user may take a snoring-event-based feedback training at step 430. Or optionally, in the case of high position dependency with high non-supine snore index, the user may combine both positional training in supine position and snoring-event-based feedback training in non-supine position. On the other hand, in the case of high snore index with lower position dependency, the user may go through step 440 to check if there is a POSA (Positional Obstructive Sleep Apnea), since according to the research, the higher a user's snore index, the more often they were found to be position independent, that means the more serious blockage of upper airway may possibly lead to OSA symptoms.

Referring to FIG. 5, the flow chart illustrates the main steps to evaluate the relation between the sleep positions and sleep respiratory events and provide a corresponding prevention method. At step 502, the wearable device is applied to the user by a wearable structure.

At step 505, once the wearable device is mounted, the controller unit initiates data collection to acquire the sleep position related information while the patient is asleep. The collected data can be transmitted to an external device via the wireless communication module, or can be saved into a memory in the wearable device first, and then transmitted to an external device for later analysis. Now referring to step 510, the sleep respiratory information is collected, the possible sensors include, but not limited to, an optical sensor, an accelerometer, a piezoelectric vibration sensor, a piezoelectric motion sensor, electrodes for detecting body resistance, a RIP sensor, an airflow sensor, a microphone, either implemented on a wearable device or an external device, such as smartphone, without limitation.

Now at step 515, both the sleep position related information and the sleep respiratory information are combined, so that a correlation may be calculated by a software program. For example, the supine sleep respiratory event index is defined as the number of respiratory information events per hour while lying in supine position, the non-supine respiratory information event index is defined as the number of respiratory information events per hour while lying in supine position, and the respiratory information event index=supine respiratory information event index+non-supine respiratory information event index. A POSA user is defined as having a supine respiratory information event index higher than their total non-supine respiratory information event index. At step 518, a pre-defined threshold is compared with, for example, the ratio of supine respiratory information event index and non-supine respiratory information event index, or other comparisons are possible. If the threshold is exceeded, the user is identified as a POSA user, and then may take a sleep position training (SPT) at step 525. Otherwise, the user may take a respiratory information event based feedback training at step 530. Or optionally, in the case of high position dependency with high non-supine respiratory information event index, the user may combine both positional training in supine position and respiratory information event based feedback training in non-supine position.

The sleep position training is that when a detected sleep position meets a predetermined position range, e.g., a supine position, and continues for a period of time (e.g., 5-10 seconds), the alarm unit activates alarms, e.g., vibrations or sounds, and the strength of alarms will increase gradually until the sleep position is out of the predetermined position range, such as changes to a different sleep position or non-supine position. Then, the alarm stops. If the sleep position doesn't change after a predetermined period of time (e.g., adjustable 10 to 60 seconds), then the alarm pauses and restarts after a predetermined period of time (e.g., adjustable several minutes). In some embodiments, the frequency/duration of the alarm is very short at the beginning and increases gradually until the user is no longer in the supine position. Further, alarms also have intervals (e.g., 2 seconds) and repeat times (e.g., 6 times).

The setting of the predetermined position range can be varied according to different demands, such as based on the definition of supine position, the predetermined position range can be different. For example, in an embodiment, when the accelerometer is deployed on the torso, the range can be set as an included angle between the surface normal of the torso and the surface normal of the bed varied from +30° to −30°. In another embodiment, when the accelerometer is deployed on the forehead, since there have more activities of the head during sleep, the range can be set as an included angle between the surface normal of forehead and the surface normal of the bed varied from +45° to −45°. In another embodiment, when the accelerometer is deployed on the neck, the range can be set as the range as deployed on the forehead.

The positional training for snore events is similar, and the only difference is the alarm is provided based on the detection of snoring. Therefore, the description is omitted.

Following is how the alarms are provided. The control unit generates a driving signal and after receiving the driving signal, the alarm unit produces at least an alarm for providing to the user, thereby achieving the purpose of sleep positional training and/or sleep respiratory feedback training. The driving signal is generated according to an alarm behavior which is decided through comparing the sleep position related information with a predetermined position range, and the sleep position related information meets the predetermined sleep position range and/or comparing the sleep respiratory information with a predetermined condition, and the sleep respiratory information meets the predetermined condition. The details are described below with embodiments.

Note that the alarm unit described above, no matter for providing which type of alarms, such as vibrations or sounds, can be embodied differently, for example, can be deployed in the wearable device for acquiring sleep physiological information, or in another wearable device, or in an external device, without limitation.

Further, the provision of alarms is preferably performed after ensuring that the user has already fallen asleep. For achieving this, in an embodiment, the present disclosure utilizes the sleep physiological information to know that if the user has fall asleep, and after ensuring the user is asleep, the system changes into an alarm producing state and starts to perform the sleep positional training and/or the sleep respiratory feedback training.

The sleep physiological information acquired by the physiological sensor is compared with a predetermined condition for deciding if the physical condition of the user meets a predetermined sleep respiratory condition. The predetermined sleep respiratory condition adopts physical conditions which only happen after asleep, for example, if the ODI event, low oxygen level event, heart rate variation sleep respiratory event, snore event, apnea event and/or hypopnea event occurs. When the physical condition of the user meets the predetermined condition, the system changes to enter the alarm producing state in which the control unit generates the driving signal for driving the alarm unit to provide alarms in accordance with the alarm behavior decided.

In an embodiment, the snoring which can be detected by the microphone or the accelerometer is adapted as the basis since snore mostly occurs before OSA happens. Accordingly, the happening of snore can be the timing for starting the sleep positional training and/or the sleep respiratory feedback training. In an embodiment, the analysis results of heart rate are adopted as the basis, for example, the specific variation of heart rate before fall asleep, and HRV which shows the body condition. In an embodiment, the respiration is analyzed to know if the user has fallen asleep, for example, the speed of breathing will become slower when asleep. In an embodiment, the sleep stage can be the basis, for example, by analyzing the actigraph acquired by the accelerometer or the heart rate acquired by the optical sensor, the sleep stage can be revealed. In still another embodiment, the sleep respiratory events also can be the basis. Therefore, all kinds of sleep respiratory information from various kinds of physiological sensors can be utilized without limitation.

Further, the physiological sensor which is utilized for acquiring the physiological information for deciding if the system enters the alarm producing state may be deployed at different locations as needed. For example, it can be the physiological sensor which is employed to perform the training, or an additional physiological sensor deployed in the wearable device for performing the training or another wearable device, such as an accelerometer, an optical, a microphone etc., or in an external device, such as a microphone located beside the bed or an accelerometer located on the bed.

FIG. 7 is the flow chart for illustrating the sleep positional training and the sleep respiratory feedback training are performed in the same sleep duration. Through deploying the position sensor and at least a physiological sensor, it is able to acquire the sleep position related information and the sleep respiratory information in the same sleep duration. Depending on which kind of sleep respiratory information to be acquired and the deploying location of the physiological sensor, the selection of the physiological sensor includes but not limited an optical sensor, a microphone, an accelerometer, a piezoelectric vibration sensor, a piezoelectric motion sensor, electrodes for detecting body resistance, a RIP sensor, and/or an airflow sensor. Particularly, when the accelerometer is selected, it also can be used as the position sensor.

Then, through a sleep respiratory information analysis program, the sleep position related information is compared with the predetermined condition for deciding the sleep respiratory events, and through a sleep position analysis program, the sleep position related information is compared with the predetermined position range. When the sleep position related information meets the predetermined position range, a first set of alarming conditions is provided, and when the sleep position related information is out of the predetermined position range, a second set of alarming conditions is provided. Further, an alarm deciding program is provided for deciding a corresponding alarm behavior according to the different set of alarming conditions. Accordingly, the control unit, based on the alarm behavior decided, generates an alarm signal, and after receiving the alarm signal, the alarm unit produces at least an alarm, thereby achieving the effect of influencing the sleep position and/or the sleep respiratory state of the user.

The first set of alarming conditions at least includes at least one of a time range criterion and a sleep respiratory event criterion. For example, the time range criterion can be implemented as being based on the absolute time, e.g., at 1:00 AM, or on a specific physiological condition, e.g., one hour after the user has lied down, fall asleep or other physiological conditions, or on a delay time, e.g., one hour after the device/system starts. Therefore, through the time range criterion, it can be selected to provide a more comfortable experience without waking up the user. Further, the sleep respiratory event criterion provides the possibility to select whether the sleep positional training and the sleep respiratory feedback training are performed in the same sleep duration, which can improve the training effect.

The second set of alarming conditions at least includes the time range criterion and the sleep respiratory event criterion. For example, when the sleep position related information is out of the predetermined position range, e.g., the user is at a non-supine position, the alarm is produced mainly based on the occurrence of sleep respiratory events, namely, the sleep respiratory feedback training. Further, as described above, the time range criterion is also applicable to the sleep respiratory feedback training, such as based on the absolute time, the specific physiological condition, or the delay time.

Furthermore, other criteria also can be used. For example, an alarm strength criterion and/or an alarm frequency criterion can be adopted, so that the alarms can be provided at a lower level of strength or with a lower frequency in the beginning and increasing after a period of time. Thus, through providing different sets of alarming conditions, the training(s) can be performed by more conforming to the practical demands without interfering the user's sleep.

In addition, since the sleep position is changed all the time during sleep, the provisions of the first set of alarming conditions and the second set of alarming conditions are dynamic and can be in any application order, namely, the provision order totally depends on the real time sleep position of user without limitation.

In the present disclosure, according to the different functions performed, the system may correspondingly include various kinds of programs, for example, sleep physiological information analysis program, sleep respiratory information analysis program, sleep respiratory event analysis program, alarm deciding program etc., so as to obtain various kinds of physiological information from the physiological signals acquired by the physiological sensors. And, without limitation, the programs can be preloaded in any suitable device.

According to the sleep respiratory feedback training based on the sleep respiratory information (as shown in FIG. 3) and the sleep related evaluations and prevention methods based on the sleep position (as shown in FIG. 4 and FIG. 5) together with all the possible placing positions of physiological sensors for acquiring related physiological signals, the present disclosure, without limitation, may have embodiments described below.

Firstly, the present disclosure is related to the evaluation of sleep positions and sleep disorders and to how to improve positional sleep disorders.

In one aspect, a dispersed deployment of system is employed to achieve a best performance.

When the dispersed deployment is adopted, how dispersed devices communicate with each other and/or with external device(s) becomes very important, which not only is related to feasibility but also convenience. The dispersed system of the present disclosure means a system including more than two devices capable of functioning independently with circuitry such as control unit, power module, communication mode etc. The communication can be implemented as wireless such that the devices can be communicated wirelessly via digital signals for providing convenience.

As described above, the conventional technologies mostly focus on a single device for monitoring physiological information and providing alarms at the same time. However, since it is preferred to acquire sleep position around the longitudinal axis of human body or at other locations where the sleep position can be obtained after calculation, there is difficulty in considering both in some situations.

When the dispersed deployment is adopted, firstly, the location for placing the alarm unit and the type of alarms can be selected freely. For example, some people may be sensitive to vibrations and others may be sensitive to sounds, or the different portions of human body may have different sensitivities to the alarms.

Further, the dispersed deployment also makes the acquisition of sleep physiological information have more possibilities. As described above, one kind of physiological information may be acquired by different kinds of physiological sensors at various body portions, and thus, the dispersed deployment can help the acquisition more close to the real demand, for example, different users may have different sleep disorder symptoms, and through selecting a physiological sensor which can correctly represent the real physical condition, the corresponding training can be more effective. Moreover, the user's feeling also matters, for example, the feeling about having a device placed on the body surface may be different for different people, and the dispersed deployment gives the user the possibility to select the body portion for placing the device with least interference.

In one embodiment, a sleep physiological system includes two devices, a sleep alarm device and a sleep physiological device. The sleep alarm device includes a first wearable structure, a first control unit which at least includes microcontroller/microprocessor, a first wireless communication module electrically connected to the first control unit, an alarm unit electrically connected to the first control unit, and a power module, wherein the first wearable structure is used to mount the sleep alarm device on a user's body, so that the alarm unit can produce at least an alarm for providing to the user. The sleep physiological device includes a second wearable structure, a second control unit which at least includes microcontroller/microprocessor, a second wireless communication module electrically connected to the second control unit, a position sensor electrically connected to the second control unit, and a power module, wherein the second wearable structure is used to mount the sleep physiological device on the user's body, so that the position sensor can acquire sleep position related information of the user during sleep for being a reference for providing the at least an alarm.

The above sleep physiological system is namely a dispersed sleep position training system. Through this deployment, the alarm unit can be selected to adopt vibrations or sounds freely and placed at any suitable body portion. Further, the position sensor is no more limited to place at a body portion where should be able to sense the alarms and can be placed at any suitable body portion.

Particularly, the sleep physiological device for acquiring sleep position can be implemented to place on the torso, e.g., the abdomen or the chest, through employing a belt or an adhered structure, or through mounting on the clothes. Since the sleep position information can be acquired without contacting the skin, the device also can be placed at the outer surface of clothes. The sleep alarm device can be implemented to locate at a body position usually used for mounting devices, for example, the wrist, the finger etc., with a popular style, such as a wrist-worn style, a finger-worn style etc., for providing vibration alarms. The cooperation therebetween maximizes the usage convenience and minimizes the burden on the user's body, e.g., the sleep physiological device can be mounted on the chest along with the sleep alarm device being mounted on the wrist.

Without limitation, the sleep physiological device also can be placed at other locations, e.g., the forehead, the neck. Identically, the sleep alarm device can be mounted on other locations by adopting other types of alarms, for example, be mounted on or around the ear by adopting audible alarms. Further, the sleep alarm device can be implemented as an earphone connected to an external device, for example, the external device communicates with the sleep physiological device and drives the earphone to provide audible alarm according to the sleep position from the sleep physiological device, or can be implemented as an earphone capable of communicating with the sleep physiological device directly. Therefore, all kinds of implements are possible without limitation.

The transmission of physiological information between devices has some options. For example, in an embodiment, both the sleep physiological information analysis program and the alarm deciding program are preloaded in the sleep physiological device, namely, the sleep position related information is compared with the a predetermined position range first and an alarm behavior is decided when the sleep position related information meets the predetermined position range. Then, the alarm behavior is transmitted to the sleep alarm device through digital signals, and after the control unit in the sleep alarm device receives the digital signals, a driving signal is generated according to the alarm behavior for driving the alarm unit to produce at least an alarm for providing to the user so as to achieve an alarm effect, such as to induce an automatic position change. This manner is advantageous for saving power of the sleep alarm device, e.g. for extending the period of battery changing.

Alternatively, the sleep alarm device also can be implemented to receive the sleep position related information and, through the preloaded programs, to perform analysis and decide the alarm behavior. In this case, the sleep position related information is first transmitted to the sleep alarm device and compared with a predetermined position range to device the alarm behavior, and then the control unit of the sleep alarm device, according to the alarm behavior, generates the driving signal so as to drive the alarm unit to produce at least an alarm for the user. Alternatively, it also can be implemented as the sleep position related information is analyzed in the sleep physiological device to know if it is within the predetermined position range, and the comparison result is transmitted to the sleep alarm device through digital signals for deciding the alarm behavior. Then, the control unit of the sleep alarm device, according to the alarm behavior, generates the driving signal for driving the alarm unit to produce at least an alarm for the user. Therefore, there are different possibilities without limitation.

There are more possibilities when involving an external device. For example, the sleep physiological information analysis program and the alarm deciding program both can be preloaded in the external device. In this case, the sleep position related information acquired by the sleep physiological device will be transmitted to the external device, and the external device perform a sleep physiological information analysis procedure and an alarm deciding procedure to decide if there is a need to provide the alarms and how to provide the alarms by deciding an alarm behavior. Then, the alarm behavior is transmitted to the sleep alarm device through digital signals, and after receiving the digital signals, the control unit of the sleep alarm device generates the driving signal according thereto to drive the alarm unit to produce alarms. Alternatively, it also can be implemented as only the sleep physiological information analysis program or only the alarm deciding program is preloaded in the external device.

Furthermore, it is preferable to employ more physiological sensor(s) for acquiring other sleep respiratory information. For one benefit, it can be used to ensure the effect of sleep position training, e.g., if the times of sleep respiratory events happened reduce, and for another benefit, it can be used as the basis for performing a sleep respiratory feedback with the sleep position training within the same sleep duration, thereby enhancing the training effects. For example, the additional physiological sensor can be mounted on the sleep physiological device and according to the position of the longitudinal axis it is placed, there have different possibilities. When the device is placed on the forehead, the physiological sensor can be the optical sensor, accelerometer, microphone and/or piezoelectric vibration sensor to acquire physiological information such as blood oxygen saturation, heart rate, snoring related information etc. When the device is placed between the nose and the mouth, the physiological sensor can be the airflow sensor, optical sensor, accelerometer, microphone and/or piezoelectric vibration sensor to acquire physiological information such as breathing flow variations, heart rate, snoring related information. When the device is placed on the torso, the physiological sensor can be the optical sensor, accelerometer, microphone, piezoelectric vibration sensor, electrodes for detecting body resistance, and/or RIP sensor. Alternatively, the additional physiological sensor also can be mounted on the sleep alarm device or the external device for acquiring physiological respiratory information according to the body position. Then, the sleep respiratory information acquired can be utilized to obtain sleep respiratory events, such as ODI event, low oxygen level event, heart rate variation sleep respiratory event, snore event, apnea event and hypopnea event.

In the following descriptions, the contents of the dispersed deployment are all similar and related to the framework of more than two independent devices communicated wirelessly. Therefore, the related procedures, e.g., information transmission between/among devices and/or with the external device, information analysis, and alarm behavior decision, all can be referred to the contents described above and are omitted for simplification.

Further, as known by ones skilled in the arts, the devices in a wireless dispersed system should be equipped with the basic circuitry, such as control unit, wireless communication module and/or wired communication module, and power module. Thus, these contents are also omitted in the following embodiments for simplification.

In another embodiment, a sleep physiological system includes two devices, a sleep alarm device and a sleep respiratory device, both mounted on a user through wearable structures. The sleep alarm device includes a position sensor for acquiring sleep position related information from the user, and an alarm unit for providing the user at least an alarm. The sleep respiratory device includes a physiological sensor for acquiring sleep respiratory information of the user in the sleep duration. In this case, because both the sleep position and the sleep respiratory information can be obtained, no matter the positional or the non-positional sleep disorders can be improved in this system, namely, this system combines both the sleep position training and the sleep respiratory feedback training, and is capable of comprehensively improving sleep breathing disorders. Advantageously, the alarm unit in the sleep alarm device can selectively produce the alarms according to different sleep physiological information, for example, according to sleep position related information, according to sleep respiratory information, or according to both the sleep position related information and the sleep respiratory information. Therefore, this system will be able to provide effective solutions for users with any type of SDB symptom or users with combined SDB symptoms, such as MSA symptom, or even users who still don't know which type of symptom he/she has.

Herein, the sleep position related information is compared with a predetermined position range, and the sleep respiratory information is compared with a predetermined condition, thereby the alarm behavior can be decided based on both or one of the comparison results.

Further, this kind of system also can be operated differently. Since the sleep alarm device itself is already equipped with the position sensor and the alarm unit, it can be selected to use alone for performing sleep position training or to cooperate with the sleep respiratory device to magnify the effectiveness. Accordingly, it is possible for the user, for example, to select how many devices will be placed on the body and which kinds of sleep physiological information will be a basis of providing alarms. These are the benefits that only the wireless system can provide.

When the sleep alarm device is implemented to place on the torso, it is preferably to adopt a vibration alarm, and when the device is placed on the forehead, it can be selected to use vibration or sound alarms.

Based on the dispersed framework, it is advantageous that different kinds of physiological sensors and different placing locations thereof and also different kinds of sleep respiratory information can be selected. Accordingly, the predetermined condition will be changed in accordance with the physiological sensor selected, and the wearable structure for carrying the sleep respiratory device also will be varied.

For example, the sleep respiratory device can be implemented to adopt a common wearable style in daily life, such as wrist-worn or finger-worn style by using optical sensor or microphone to acquire sleep respiratory information, e.g., heart rate, blood oxygen saturation, respiratory behavior, snoring related information and variations of breathing sounds, and thus, the wearable smart device, such as smart watch, smart ring, smart earphones, will be suitable for this case. Further, the sleep respiratory device also can be implemented to locate near the user and not place on the user's body, e.g., the microphone of a smartphone can be used to detect the sounds of snoring and breathing so as to obtain the sleep respiratory information. Then, depending on the type of sleep respiratory information acquired, various kinds of sleep respiratory events can be obtained through the sleep respiratory event analysis program, e.g., ODI event, low oxygen level event, heart rate variation sleep respiratory event, snore event, apnea event and hypopnea event. Accordingly, it only needs to cooperate with the sleep alarm device which may be placed on the torso/head/neck to detect sleep position and provide vibration alarm, such that the system capable of providing two kinds of trainings can be integrated with the common used device(s), for example, the sleep alarm device can be placed on the forehead along with the sleep respiratory device placed on the finger, or the sleep alarm device can be placed on the back of neck along with the smartphone being the sleep respiratory device. The concept of integrating the smart device is beneficial to the popularity of this system.

In an embodiment, a sleep physiological system includes two devices, a sleep alarm device and a sleep respiratory device, and both are mounted on a user's body through wearable structures. The sleep alarm device includes an alarm unit for providing at least an alarm to the user, and the sleep respiratory device includes a physiological sensor for acquiring at least one kind of sleep respiratory information of the user in a sleep duration. Through wireless communication, the acquired sleep respiratory information is implemented to be the basis for the alarm unit to produce alarms. The sleep respiratory information is utilized to obtain at least a sleep respiratory event so as to decide an alarm behavior, and a driving signal generated according to the alarm behavior will drive the alarm unit to produce the at least an alarm to the user so as to achieve an alarm effect, such as cause the user to briefly awaken and breath normally.

The sleep physiological system is namely a dispersed sleep respiratory feedback system. Through the framework thereof, the alarm unit can be selected to adopt tactile or audible alarms and can be placed at any location that is suitable for sensing the alarms. Further, the types of physiological sensor and the sleep respiratory information also can be selected freely. Since sleep disorder symptom varies from different users and the physiological sensors therefor change accordingly, the dispersed design makes the system have a broader application range with more flexibility. For example, the physiological sensor can be implemented to be, e.g., optical sensor, accelerometer, microphone, piezoelectric motion sensor, piezoelectric vibration sensor, electrodes for detecting body resistance, and/or RIP sensor, placing on, e.g., head, ear, neck, torso, wrist, and/or finger, for acquiring sleep respiratory information, e.g., snoring related information, variation of breathing sounds, respiratory effort, variation of breathing flow, respiratory behavior, heart rate, and/or blood oxygen saturation, so as to decide various kinds of sleep respiratory events, e.g., ODI event, low oxygen level event, heart rate variation sleep respiratory event, snore event, apnea event and/or hypopnea event.

In addition, the sleep respiratory device can further include a position sensor for acquiring sleep position related information, and accordingly, the system becomes capable of performing the sleep position training and/or the sleep respiratory feedback training. In this case, it should be noted to place the sleep respiratory device on locations capable of acquiring the sleep position related information, such as head, neck, torso etc.

Particularly, in a preferred embodiment, the sleep alarm device can be selected to adopt a tactile alarm and place on the wrist for convenience. Even more, it will be able to directly employ the wearable device, which provides the function of vibration, in the market, such as smart watch, smart ring etc., as the sleep alarm device and further to utilize the information providing interface of the wearable device to provide various information during the performing duration. Alternatively, it is also possible to utilize the information providing interface of a smartphone without limitation. This framework will be an extremely cost effective solution for the user.

In an embodiment, a sleep physiological system includes two devices, a first sleep physiological device having a first sleep physiological sensor for acquiring a first sleep physiological information and a second sleep physiological device having a second sleep physiological sensor for acquiring a second sleep physiological information. Further, at least an alarm unit is implemented to mount in the first sleep physiological device and/or the second sleep physiological device for providing alarms according to the sleep physiological information. Through wireless communication, the alarm unit can be implemented to provide alarms according to the first sleep physiological information, the second sleep physiological or the first and the second sleep physiological information.

The first sleep physiological device and the second sleep physiological are both wearable devices, and according to the placing locations thereof, the sleep physiological sensor employed and the sleep physiological information acquired will be different. For example, the placing location includes, but not limited, head, neck, torso and upper limb. The sleep physiological sensor employed includes, but not limited, optical sensor, accelerometer, airflow sensor, piezoelectric motion sensor, piezoelectric vibration sensor, electrodes for detecting body resistance, RIP sensor, microphone, EEG electrodes, EOG electrodes and EMG electrodes. The sleep physiological information acquired includes, but not limited, snoring related information, variation of breathing sounds, respiratory effort, variation of breathing flow, respiratory behavior, heart rate, blood oxygen saturation, EEG signals, EOG signals, EMG signals, sleep position, sleep actigraph and sleep stage. The sleep respiratory event obtained includes, but not limited, ODI event, low oxygen level event, heart rate variation sleep respiratory event, snore event, apnea event and hypopnea event. Namely, in this embodiment, the basis for deciding the alarm behavior is unlimited, e.g., the basis can be the snoring related information plus the blood oxygen saturation, the heart rate plus the blood oxygen saturation, or the sleep position plus the respiratory effort. Another possibility is the sleep physiological information is used to decide the alarm behavior and the other is used to monitor the physical state of the user in the sleep duration. Therefore, there are various possibilities.

For example, in a preferred embodiment, the first sleep physiological device is placed on the wrist with optical sensor, accelerometer and/or microphone to acquire heart rate, respiratory effort, snoring related information, variation of breathing sounds, sleep actigraph and/or sleep stage, and the second sleep physiological device is placed on the finger with the optical sensor to acquire blood oxygen saturation, in a result that two sleep physiological information can be acquired on the same upper limb.

Other than the embodiment above, the first sleep physiological device and the second sleep physiological device also can be placed on any other location by wearable structure, such as head, ear, torso, arm, wrist, and finger, so as to employ identical or different physiological sensors to acquire more sleep physiological information.

Particularly, if one of the first sleep physiological device and the second sleep physiological device is implemented to acquire the sleep position, the alarm unit can be implemented to provide alarms according to the sleep position and/or the sleep respiratory information, so as to perform the sleep position training and/or the sleep respiratory feedback training. If both the first sleep physiological device and the second sleep physiological device are implemented to acquire sleep respiratory information, the alarm unit can be implemented to provide alarms according to at least one of these two sleep respiratory information, so that two information can be complement for each other.

When the wearable structure is implemented to be similar to that of the wearable smart device, such as wrist-worn type or ear-worn type, it is also possible to conveniently adopt the wearable smart device to achieve the functions/behaviors described above. Further, the one which is equipped with the alarm unit of the first sleep physiological device and the second sleep physiological device can be selectively to independently perform the sleep training, or to cooperate with another device for providing more functions.

In addition, except for performing the training for improving the sleep disorder, the dispersed system also can be applied to evaluate the sleep disorder for providing more accurate evaluation results.

In an embodiment, a sleep physiological system includes two devices, a sleep physiological device and a sleep respiratory device. The sleep physiological device includes a position sensor and is mounted on a user's body, and the sleep respiratory device includes a physiological sensor for acquiring sleep respiratory information. Through the dispersed deployment, both the sleep position related information and the sleep respiratory can be accurately acquired from a suitable location. It is advantageous that the provision of sleep respiratory information can have flexibility to meet different physical conditions, for example, without being limited to the locations for acquiring sleep position related information, the sleep respiratory event can be selected to be snore event, ODI event or other suitable events, and no matter which event is selected, the evaluation can be performed accurately. Then, through being analyzed with the sleep position, it will be able to decide a ratio of sleep respiratory events happened when sleep positions meet the predetermined position range to sleep respiratory events happened when sleep position doesn't meet the predetermined position range, e.g., the ratio of supine to non-supine. Therefore, a sleep respiratory event position correlation information can be provided to the user, e.g., through the information providing interface, so as to understand that the correlation between the sleep respiratory event and the sleep position is high or low.

Similarly, in this framework, the smart device also can be utilized as the sleep respiratory device for detecting the sleep respiratory information, for example, through the optical sensor and/or microphone of the smart watch or the microphone of the smartphone. Since this system emphasizes on evaluating if the user has sleep disorder and the relation thereof with the sleep position, the provision of information is important, and thus, the existing information providing interface of the smart device can be used directly, e.g., the screen, LED and/or sounding elements of the wearable smart device or the smartphone. This not only is simple and convenient, but also matches the daily behavior of the user.

For example, in practice, the sleep physiological device can be mounted on the torso along with the sleep respiratory device being mounted on the finger for utilizing the optical sensor to acquire blood oxygen saturation and further calculating ODI, or along with the sleep respiratory device being mounted the wrist for utilizing the optical sensor to acquire average blood oxygen saturation, heart rate, and/or respiratory behavior, or along with a microphone for acquiring the snoring related information, so that the relationship between the occurrence of sleep respiratory event and the sleep position can be revealed. Further, ear is also a location suitable for mounting physiological sensor, for example, the optical sensor can be mounted on the ear and according to different portions of the ear, the acquired PPG signals can be analyzed to obtain blood oxygen saturation or to obtain heart rate and respiratory behavior, the microphone can be mounted to acquire snoring sounds, or the accelerometer can be mounted to detect the vibrations caused by snoring. In addition, the airflow sensor also can be used to mount between the nose and the mouth to know if there any sleep apnea and/or sleep hypopnea happened. Therefore, there are various possible locations for mounting the physiological sensor without limitation.

A wearable structure can be utilized to mount the device on the body, for example, an adhesive structure, a belt, a head-worn structure, a finger-worn structure, a wrist-worn structure, and an ear-worn structure. It is also appropriate to adopt two wearable structures at the same time without limitation.

Furthermore, the system can further include an alarm unit, e.g., mounted in the sleep physiological device and/or the sleep respiratory device, for improving sleep disorders. For example, if it is found that the ratio of sleep respiratory events happened in the period of supine position is higher, the alarms can be implemented to focus on the supine position, e.g., through vibrations produced by a vibration module, so as to cause a spontaneous change of sleep position, thereby improving the positional sleep disorders. Alternatively, it also can be implemented to provide the alarms when sleep respiratory events obtained by analyzing the sleep respiratory information, such as snore events or ODI events, happen, so as to perform sleep respiratory feedback training. In such case, this system becomes to combine both the evaluation and the training. For example, at beginning, the user can select to not provide alarms but to use the two devices to evaluate if there are sleep disorders happened in the sleep duration and the relationship thereof with the sleep position, and then, when it is found that the correlation between sleep respiratory events and sleep position is high, e.g., there is high ratio of sleep respiratory events happened during supine position, this system can further to provide the function of sleep position training, or if the correlation therebetween is low, this system also can provide the function of sleep respiratory feedback training. That means only one system can provide multiple functions with great benefits.

In an embodiment, a sleep physiological system includes two devices, a sleep alarm device and a sleep respiratory device. The sleep alarm device includes a position sensor mounted on the user's body and an alarm unit for providing at least an alarm to the user, and the sleep respiratory device includes physiological sensor for acquiring sleep respiratory information in a sleep duration of the user. In this framework, firstly, the sleep alarm device can be used independently for providing alarms, namely, for providing the sleep position training, and then, when the sleep alarm device is cooperated with the sleep respiratory device, the sleep respiratory information acquired by the sleep respiratory device can be used to verify the effect of alarm provision, e.g., if the occurrences of sleep respiratory events, such as sleep apnea and/or snore event, are reduced due to the changes of sleep position, thereby the user can clearly know that if the training works and how's the effect thereof, e.g., through the information provided by the information providing interface, such as the numbers and occurring time of alarming, the numbers and occurring time of sleep respiratory events, and the distribution and ratio of different sleep positions.

For understanding the difference before and after the sleep position training, at first, the sleep alarm device can be implemented not to provide alarms but to detect the sleep positions in the sleep duration along with the sleep respiratory device to acquire the sleep respiratory information, such that the relationship between the occurrences of sleep respiratory events and the different sleep positions can be revealed. Then, when it is started to perform the sleep position training, the sleep respiratory information can be used to realize the effect of alarm provision, for example, if the ratio of different sleep positions varies, and if the occurrence of sleep respiratory events reduces.

Through this framework, a long term, e.g., daily, monitoring for sleep position training can be achieved. Accordingly, the alarm behavior can be adjusted through the long term acquired sleep physiological information during training, so that the provision of alarms can be more effective, and the interference for the user's sleep can be minimized.

Since the sleep alarm device is equipped with the position sensor and the alarm unit at the same time, when the user has ensured his/her sleep disorder has high correlation with the sleep position and also the alarm provision is effective, then the user can select to use the sleep alarm alone for simplifying the deployment on the body. Then, once a period of time, e.g., every month, the user can again use two devices for checking if the physical condition changes and adjusting the alarm behavior accordingly, so as to keep the effect of sleep position training. Further, because human body may be used to a certain sleep position, e.g., be used to the supine position after a period of training, it can try to stop the sleep position training and only perform the detection of sleep position and/or sleep respiratory information, so as to obtain more information for adjusting.

In practice, for example, the sleep alarm device mounted on the torso, head or neck can cooperate with the sleep respiratory device mounted on the finger to acquire blood oxygen saturation and ODI through optical sensor, or with the sleep respiratory device mounted on the wrist to acquire average blood oxygen saturation, heart rate, respiratory behavior etc. through optical sensor, so as to check and/or perform the sleep respiratory feedback training. Further, ear is also a location suitable for mounting physiological sensor, for example, the optical sensor can be mounted on the ear and according to different portions of the ear, the acquired PPG signals can be analyzed to obtain blood oxygen saturation or to obtain heart rate and respiratory behavior, the microphone can be mounted to acquire snoring sounds, or the accelerometer can be mounted to detect the vibrations caused by snoring. In addition, the airflow sensor also can be used to mount between the nose and the mouth to know if there any sleep apnea and/or sleep hypopnea happened. Therefore, there are various possible locations for mounting the physiological sensor without limitation.

Except for the above embodiments for obtaining physiological information and providing alarms, the following are the other details of the dispersed deployment of the present disclosure.

There are many options for information provision. For example, the information providing interface can be mounted on one or both of the two devices, or an external device, such as a smartphone, a smart watch, can be used as the information providing interface. The contents of provided information also have many possibilities, e.g., sleep position related information, sleep physiological information, sleep respiratory information, sleep respiratory events, alarm behavior, the effect achieved by alarms, the times for providing alarms etc. All kinds of information during sleep can be provided to the user through the information providing interface without limitation.

Under the dispersed framework adopting wireless communication, it should also pay attention to the operations between two devices and the integration of physiological information obtained by different devices.

The operations of the system, such as the start/stop, parameter settings, may vary differently. For example, it can be implemented to use the external device wirelessly communicated to operate, e.g., through the application loaded in the smartphone, such that the operation interface thereof can be used to control the system. It also can be implemented to have an operation interface mounted on one of the devices for controlling the other device through wireless communication. Furthermore, how to start the system also can be different. For example, except for starting through the operation interface, it also can start automatically, e.g., when the deployment on the body is detected, or at a preset time.

As to the storage of information, it can be selected to store the physiological information in each device which acquires thereof, and in this case, each device may be equipped with a data storage unit, such as memory. Further, it also can be selected to store the information in one single device, for example, the physiological information acquired by one of the devices is wirelessly transmitted to and stored in the other device. Then, at the end of sleep, the stored information can be sent out, e.g., to an external device, such as a cell phone, a computer, through wireless communication, e.g., Bluetooth, or wired communication, e.g., USB interface, or by employing a memory card. On the other hand, it also can be selected to wirelessly transmit and store the information acquired by both devices to and in the external device in real time; or alternatively, the information acquired by one device can be transmitted to the other device first and then transmitted to the external device along with the information acquired by other device.

Because the physiological information is acquired by two devices, for effectively utilizing the information, it is very important to align timelines between multiple information.

For example, the timeline alignment between the alarms provisions and the sleep positions is the basis for confirming the effects of alarm provisions, e.g., through the comparison therebetween, it can know that if the provision of alarms changes the sleep position and how are the effects of alarming strength, frequency and/or mode on the change of sleep position. Further, the relationship between the acquired physiological information and the sleep positions is the basis to judge if the sleep disorder is positional, e.g., through analyzing the physiological information, it can reveal if the sleep respiratory event happened and further confirm what kind of sleep position is when the sleep respiratory event happened. Therefore, for the dispersed sleep physiological system of the present disclosure, the alignment of timelines among all kinds of physiological information is the basis of analysis and operation.

There are many options for aligning timelines. For example, it can select to utilize time stamps for aligning timelines so as to integrate information, or it can select to perform a time synchronization before the system starts to operate. It is preferable to integrate the alignment procedure with the initiation operation for the system, e.g., when the start button of the system is pressed or the system is wirelessly initiated by the external device, so as to make the operation more convenient.

Noted that although the above embodiments are described with two devices, the dispersed framework of the present disclosure is not limited thereby and can be implemented to employ more devices, such as three or four devices, according to the practical demands.

Following, in another aspect of the present disclosure, it is related to adopting one signal device to different locations for providing multiple functions. Namely, the same device is configured to be capable of placing on at least two locations of the body through combining with different wearable structures or utilizing the same wearable structure.

For evaluation of sleep disorder, an embodiment is a sleep physiological system including a housing and at least a wearable structure. Through the at least a wearable structure, the housing can be mounted at different body positions, such as a first body position and a second body position. When two wearable structures are employed for mounting at different body portions, the housing is further implemented to be removable from the wearable structures for facilitating exchanging. Further, the sleep physiological system further includes, in the housing, a control unit at least including microcontroller/microprocessor, a position sensor electrically connected to the control unit, at least a physiological sensor electrically connected to the control unit, a communication module, and a power module, wherein when being mounted on the first body portion, the position sensor and the at least a physiological sensor respectively acquire the sleep position related information and the sleep respiratory information, such that through analyzing and comparing two kinds of information, a sleep respiratory event position related information can be obtained for revealing the relationship between the sleep position and the sleep disorder. Accordingly, the first body portion is a location near the longitudinal axis of the body, e.g., torso, head, neck ect. When the device is mounted at the second body portion, the at least a physiological sensor acquires sleep respiratory information, such that there is no limitation to the second body portion and it can be any location capable of acquiring physiological information, e.g., the head, torso, upper limbs, lower limbs etc.

It is advantageous that the user can decide how to use the device according to his/her demands without being limited by a fixed mounting location. The general physiological devices, especially those mounted through the wearable structure, mostly have only one mounting location, such as ring, watch, wristband, so that if the user has different physiological monitoring demands, for example, monitoring during sleep and in daily life, the user usually only has to buy another physiological device, which is uneconomic.

Through this system of the present disclosure, when the housing is mounted on the first body portion, both the sleep respiratory information and the sleep position related information can be acquired, so that except for acknowledging if the user has sleep disorder, an evaluation for positional sleep order is also possible, which provides the ability of distinguishing the category of sleep disorder and is especially practical for the situation that a high ratio of sleep disorders are positional sleep disorders. Furthermore, because there is no limit to the second body portion, it can be selected to be a location most easily mounted, e.g., the wrist, for understanding the respiration during sleep. Therefore, for example, at the beginning, the housing can be mounted on the second body portion for acquiring the sleep respiratory information to make sure that if there have sleep disorders happened, and then, if it has confirmed the happening of sleep disorders, the housing can be moved to the first body portion where both the sleep respiratory information and the sleep position related information can be acquired for further confirming that if the sleep disorders are positional sleep disorders.

The selections of the at least a physiological sensor and the body portion have many possibilities. For example, it can select to use the optical sensor for acquiring blood physiological related information, such as blood oxygen saturation, heart rate and/or respiratory behavior, and in this case, the first body portion can be torso, forehead etc., and the second body portion can be finger, wrist, arm, ear etc. Alternatively, it can select to use the microphone for acquiring snoring related information and/or the variations of breathing sounds, and in this case, the first body portion can be torso, forehead etc. and the second body portion can be finger, wrist, arm, ear etc. Alternatively, it can select to use the accelerometer, wherein the first body portion can be torso, forehead etc. for acquiring physiological information, such as heart rate, snoring related information, respiratory effort etc., and the second body portion can be finger, wrist etc. for acquiring heart rate. Particularly, when the accelerometer is selected to be the physiological sensor, it also can be used as the position sensor at the same time for simplifying the manufacturing procedure and reducing the cost. Thus, there can be all kinds of possibilities.

There are other options for the second body portion. In some embodiments, since the location is not limited, it is also suitable for daytime usage, e.g., the body portions, such as finger, wrist and ear are all suitable for both sleep and daytime usages. For example, the optical sensor can be used to acquire blood oxygen saturation, heart rate and respiratory behavior, and the accelerometer can be used to provide sleep actigraph, sleep stage and daytime actigraph. Further, in some embodiments, if users are using products which can help for sleep or solving sleep disorders, such as anti-snoring pillow and chin belt, the physiological information acquired can be used to realize the effect thereof. Therefore, through the possibility to change the location, the system of the present disclosure provides multiple functions by single housing, which significantly promotes the user to use this system.

There are many possible combinations of the selections of physiological sensor/position sensor and the first body portion/the second body portion and are not limited by the embodiments described above. Other combinations and selections are all within the range of the present disclosure.

Furthermore, the sleep physiological system also can additionally include an alarm unit for further providing the function of improving sleep disorder. For example, in some embodiments, when the housing is mounted on the first body portion, since both the sleep position and the sleep respiratory information can be acquired, except for performing the sleep position training through the alarm unit, the sleep respiratory information also can be used to monitor the effect of sleep position training, e.g., if the symptom of sleep disorder is reduced due to a reduction ratio of the supine position, or the sleep respiratory information further can be used as the basis to adjust the alarm behavior, e.g., the parameter settings. In some embodiments, when the housing is mounted on the first body position, the alarm unit can further perform the sleep respiratory feedback training based on the sleep respiratory information acquired, such that the alarm provision can implemented to base on the sleep position, the sleep respiratory information or both for performing the sleep respiratory feedback training and/or the sleep position training. Further, without limitation, when the housing is mounted on the second body portion during sleep, it also can be implemented to perform the sleep respiratory feedback training based on the sleep respiratory information acquired.

Here, according to different demands, the alarm unit can be, for example, mounted in the housing, mounted in another wearable device, such as a smart watch or a smart band, or mounted in an external device, such as a smartphone without limitation. Accordingly, the type of alarms used also can be different. For example, when being mounted on or near the ear, audible alarms are preferable, when being mounted on the torso, the neck or the limb (including finger, wrist and arm), vibration alarms are preferable, or when being mounted on the head, both audible and vibration alarms are preferable; or it can be implemented that the user select the preferred type of alarms. In an embodiment, the alarm unit can be implemented to be an earphone that is driven by another device (such as the smartphone, the smart watch or the smart band) for providing audible alarms.

In an embodiment, a sleep physiological system includes a housing and at least a wearable structure for respectively mounting the housing at a first body position and a second body position, a control unit at least including microcontroller/microprocessor, a first physiological sensor and a second physiological sensor electrically connected to the control unit for respectively acquiring different physiological information at the first body portion and the second body portion, a position sensor electrically connected to the control unit, a communication module, and a power module.

In an embodiment, the first body portion is the torso, the head or the neck, the first physiological sensor is the snore detector, such as the accelerometer or the microphone, the second body portion is the finger, the wrist or the arm, and the second physiological sensor is the optical sensor. In this case, it is advantageous that when being mounted on the first body portion, the system can acquire both the snoring related information and the sleep position related information, so as to obtain the relationship between the snoring and the sleep position, thereby revealing the snore events and also if the snore events are positional snore events and thus providing the user a snore sleep position correlation information. When being mounted on the second body portion, the optical sensor can acquire blood physiological related information, such as blood oxygen saturation, heart rate and respiratory behavior, and through analyzing the blood physiological related information, it can know that if the blood physiological related sleep respiratory events (such as ODI events, low oxygen level events, heart rate variation sleep respiratory events) happened in the sleep duration. Namely, through this system, the most common snore events and blood physiological related sleep respiratory events both can be monitored so as to provide the user a maximized convenience. Identically, if the snore detector is implemented to be the accelerometer, the accelerometer can be used as the position sensor for simplifying the manufacturing procedure and reducing the cost.

In the embodiments described above, the provision of various kinds of information is achieved by the information providing interface. The information providing interface can be mounted on the housing or can be implemented through the external device via a wired or wireless communication by the communication module.

In another aspect of the present invention, it provides a simplest way to acquire sleep physiological information capable of deciding various sleep respiratory events and the relationship between the events and the sleep position at one body portion.

In an embodiment, a system physiological system includes a housing and a wearable structure for mounting the housing on a user's body. The system physiological system further includes a control unit at least including microcontroller/microprocessor, a communication module, and a power module. For acquiring sleep physiological information, it is achieved by a position sensor and a physiological sensor electrically connected to the control unit, wherein the position sensor is used to acquire the sleep position related information in the sleep duration and the physiological sensor is used to acquire the snore related information in the sleep duration. Particularly, since the best position for acquiring sleep position is the torso and the neck above the torso, if the physiological sensor is implemented as the accelerometer, it is also able to provide the snore related information through detecting the vibrations of the body cavity. It is especially preferable that when the snoring is detected through the accelerometer, the detection result is not influenced by the environmental sounds, and even though the accelerometer is covered by cloth or quilt, the detection still can be performed normally with convenience.

Accordingly, through the acquired sleep position related information and the snore related information, a snore sleep position correlation information can be obtained. It is advantageous that only one single device is needed to be mounted on the torso or the neck, and it can know if there any snoring happened and the relationship between snoring and the sleep position, such as the ratio and distribution of snoring in different sleep positions. Therefore, this is a simple and effective selection for home monitoring. Particularly, when the accelerometer is selected to be the physiological sensor, it also can be used as the position sensor at the same time for simplifying the manufacturing procedure and reducing the cost. Thus, there can be all kinds of possibilities.

When the accelerometer is mounted on the torso, except for the snore related information, as described above, other sleep respiratory information also can be acquired, e.g., respiratory effort and heart rate. Further, it also can be implemented to equip additional physiological sensor, such as optical sensor, so as to acquire sleep physiological information from the skin of the torso or the neck, such as sleep respiratory events, sleep respiratory information, respiratory behavior, and/or sleep stage, such that the results can more accurate through a comparison among multiple sleep physiological information.

In addition, the system also can include the alarm unit for performing sleep positional training and/or sleep respiratory feedback training. For example, the acquired sleep position related information can be compared with the predetermined position range for deciding an alarm behavior as the predetermined position range is met and performing the sleep position training; or the acquired sleep respiratory information can be compared with the predetermined condition for deciding an alarm behavior as the predetermined condition is met and performing the sleep respiratory feedback training; or both information can be used for providing the sleep position related training and the sleep respiratory feedback training appropriately in the same sleep duration.

As to how the alarms are provided, the control unit generates a driving signal and after receiving the driving signal, the alarm unit produces at least an alarm for providing to the user, thereby achieving the purpose of sleep positional training and/or sleep respiratory feedback training, wherein the driving signal is generated according to the alarm behavior decided as described above. Further, as known by ones skilled in the arts, for operation, the device/system should be equipped with the basic circuitry, such as control unit, communication module, and power module, and these contents which are duplicate are omitted in the following embodiments for simplification.

In an embodiment, a sleep physiological system includes a housing and a wearable structure for mounting the housing on a user's body. For acquiring sleep physiological information, it is achieved by a position sensor and a physiological sensor, wherein the position sensor is used to acquire the sleep position related information in the sleep duration, and the physiological sensor is implemented to be the optical sensor for acquiring the blood physiological related information in the sleep duration. Particularly, since the best position for acquiring sleep position is the torso and the neck above the torso, the optical sensor is also implemented to acquire the blood physiological related information, such as the heart rate, from the skin of the torso and the neck. Particularly, as described above, the heart rate can be analyzed to get the information related to sleep stages, e.g., through observing heart rate variations, through calculating HRV, through performing HHT (Hilbert-Huang transform) or through other suitable methods. After realizing the variation of sleep stages, such as the ratios of deep sleep and non-deep sleep, a sleep quality related information can be obtained. This information is helpful since the effect of sleep position training is achieved by changing the sleep position through alarm provision so as to reduce the sleep apnea/hypopnea, the observation of sleep stage/sleep quality can help the adjustment of parameters for alarm provision and thus make the training more comfortable.

When the position sensor is implemented to be the accelerometer, since the accelerometer also can acquire the actigraph during sleep, the actigraph and the blood physiological related information can be analyzed together for obtaining more accurate sleep stage related information. Further, the blood related information also can be used to obtain other sleep physiological information, such as sleep respiratory information, sleep respiratory events, heart rate variability, and arrhythmia.

In this case, if the alarm unit is also equipped, the sleep position related information can be compared with the predetermined position range for deciding the alarm behavior as the predetermined position range is met, so as to provide alarms and perform sleep position training. Since the blood physiological related information can be acquired continuously during sleep, it may be used to confirm the improving effect of alarm provision, e.g., if the happening of sleep respiratory events is reduced due to the change of sleep position, so that the user can get more information through the information providing interface, e.g., the number and timing of alarm provisions, the variations of sleep position, the ratios of different sleep positions, and the number and timing of sleep respiratory events etc. Thus, the user can clearly know if the training is effective and how the effect is, and accordingly, the blood related information can be the basis for adjusting the alarm behavior, which not only improves the alarm provision, but also minimizes the interference to user's sleep.

For understanding the difference before and after the training, it also can be implemented, at the beginning, the alarm unit doesn't provide alarms and the acquired sleep position and blood related information are cooperated to know the happening of sleep respiratory events and the relationship between thereof and different sleep positions. Then, when the training starts, the information about if the alarm provision is effective can be obtained, e.g., the variation of ratios of different sleep positions and if the happening of sleep respiratory events is reduced.

In addition, the alarm behavior also can be decided according to the sleep position related information and/or the blood physiological related information, that is, it can be selected to perform the sleep positional training, the sleep respiratory feedback training or both in one sleep duration.

In an embodiment, a sleep physiological system includes a housing and a wearable structure for mounting the housing on a user's forehead. For acquiring sleep physiological information, it is achieved by a position sensor and an optical sensor, wherein the position sensor is used to acquire the sleep position related information in the sleep duration, and the optical sensor is used to acquire the blood physiological related information, e.g., blood oxygen saturation and heart rate, in the sleep duration from the user's forehead. Further, the system also includes an alarm unit for performing the sleep position training and/or the sleep respiratory feedback training according to the sleep position related information and/or the blood physiological related information.

This kind of system provides various advantages. For example, the alarm unit can be implemented to provide alarms according to the sleep position related information, and in this case, the blood physiological related sleep respiratory events, e.g., ODI event, low oxygen level event, and heart rate variation sleep respiratory event, obtained from the blood physiological related information can help the user to understand the sleep respiration thereof during sleep position training, e.g., the distribution of sleep respiratory events in different sleep positions, so as to provide a blood physiological related sleep respiratory event position correlation information, e.g., ODI event position correlation information, and also to understand the training effect, e.g., if the number of sleep respiratory events happening during training is reduced due to the change of sleep position. Moreover, the alarm unit also can be selected to provide alarms according to the sleep position related information and the blood physiological related information, so as to perform the sleep position training and the sleep respiratory feedback training in the same sleep duration for providing comprehensive effect. In addition, it also can be selected not to provide alarms at first but acquire sleep physiological information for deciding if there is any sleep respiratory event happened and the correlation between the happening of sleep respiratory events and the sleep position, and then, to select which training is performed according to the deciding results.

Most important is, for the user, a simple deployment on the forehead can provide all kinds of functions and selections described above, e.g., the evaluation of physical condition, the improvement of sleep disorder and the function selection based on demands. In Particular, the variation of blood oxygen saturation which is one of the most used physical parameters for deciding sleep respiratory events can be obtained in the simplest deployment to achieve most effective results.

Furthermore, other physiological sensor(s) also can be used. For example, the accelerometer or the microphone can be used to acquire the snore related information for being the basis of alarm provision, so as to perform the sleep position training and/or sleep respiratory feedback training according to snoring. In particular, the accelerometer also can be used as the position sensor for simplifying the manufacturing procedure and reducing the cost. It also can be implemented to mount EEG electrodes, EOG electrodes and/or EMG electrodes for acquiring EEG signals, EOG signals and/or EMG signals and, through analyzing thereof, sleep state/stage and sleep cycle can be revealed so as to provide the distribution of sleep respiratory events in each sleep stage and the relationship between the sleep position and the sleep stage.

Since the mounting position is forehead, except for head band and/or adhesive structure, the wearable structure particularly can be implemented as an eye mask. Generally, when wearing the eye mask, at least a portion of the forehead may be covered thereby, so that it only needs to mount the housing at a position capable of contacting the forehead, the optical sensor can acquire the blood physiological related information. Further, the eye mask also can help the user to fall asleep. Besides, there are more types of alarms can be selected on forehead, e.g., it can be implemented to be tactile, audible and/or visual alarms. In some embodiments, the number of housing can be increased, e.g., as two or more electrically connected housings, which not only can reduce the volume of each housing, but also can fit the curve of forehead more.

When there is the need to provide information to the user, it can be selected to utilize the information providing interface, or to utilize a communication module, e.g., a wireless module, such as a Bluetooth, BLE, Zigbee, WiFi, RF, or a wired communication module, such as USB interface, UART interface, for transmitting to another wearable device, e.g., wearable smart device, or to an external device, e.g., smartphone, tablet, personal computer or other devices capable of receiving information with the information providing interface.

In an embodiment, a sleep system includes a housing and a wearable structure for mounting the housing on a user's body. For acquiring sleep physiological information, a position sensor, a first physiological sensor and a second physiological sensor are provided. The position sensor is used to acquire sleep position related information, and two physiological sensors are used to acquire two kinds of sleep respiratory information. The first physiological sensor is configured to acquire the snore related information for obtaining snore events, and the second physiological sensor is configured to acquire the blood physiological related information for obtaining the blood physiological related respiratory events, and both events are provided to the user through the information providing interface.

As described above, sleep disorders include snoring and sleep apnea/hypopnea, so that if the information of both kinds of sleep disorders can be provided, it will be convenient for the user. In particular, snoring is generally regarded as the precursor of sleep apnea/hypopnea and also the happening of sleep apnea/hypopnea is always accompanied with snoring. For example, in one case, the gradually narrowed upper airway causes the breathing sounds to become heavier, then snoring and finally the sleep apnea/hypopnea, and in another case, after sleep apnea happened and when breathing recovers, snoring also happens, so that these two physiological phenomena can be used as the bases for confirming the happening of sleep apnea/hypopnea. Further, when the blood physiological related information, e.g., ODI, heart rate variation, low oxygen level, is used to obtain the sleep respiratory events, the body activities may cause artifacts in the physiological signals thereof and thus misjudgement. Therefore, through the correlation between two kinds of physiological information, the misjudgement can be reduced effectively.

Accordingly, through observing the blood physiological related information and the snore related information, when a predetermined set of conditions is met, e.g., the time sequence of both information is met, the happening of blood physiological related respiratory events can be judged more accurately.

Under this premise, when selecting the mounting location for the housing, the acquisition of sleep position is most considerable, so that it is preferable to select head or torso. When being mounted on the torso, snoring can be acquired by, e.g., the accelerometer through detecting the vibration of body cavity, or the microphone through detecting the snoring sounds, and the sleep apnea/hypopnea can be monitored by, e.g., the optical sensor to obtain blood physiological related information, such as heart rate. When being mounted on the head, the accelerometer and/or the microphone also can be used to acquire snore related information, and the sleep apnea/hypopnea can be monitored by the optical sensor to obtain blood physiological related information including blood oxygen saturation and heart rate. Therefore, according to the blood physiological related information, the blood physiological related sleep respiratory events, such as ODI events, low oxygen level events, heart rate variation sleep respiratory events, can be obtained.

Particularly, when being mounted on the head, except for head band and/or adhesive structure, the wearable structure can be implemented as an eye mask, which especially can help the user to fall asleep. The forehead is one of the selections for mounting the position sensor along with the covering area of eye mask is suitable for place physiological sensor, e.g., the optical sensor, EEG electrodes, EOG electrodes and EMG electrodes, so that utilizing the eye mask to be the wearable structure is particularly suitable in this embodiment.

Then, by comparing the obtained sleep respiratory events with the sleep position related information acquired by the sleep position, it can obtain the distributions of snore events and blood physiological related respiratory events respectively corresponding to the predetermined sleep position range is met or not, e.g., a position related snore index, a number of position related snorings, a duration of position related snorings, a position related apnea index, a number of blood physiological related sleep respiratory events that are related to sleep position, a duration of blood physiological related sleep respiratory events that are related to sleep position. Through these information, the user can understand the sleep disorders thereof are related to snoring or sleep apnea/hypopnea and also the relationships between the happenings of different kinds of sleep disorders and the sleep positions.

Further, when EEG electrodes, EOG electrodes and/or EMG electrodes are additionally mounted as mounted on the head, through analyzing EEG signals, EOG signals, and/or EMG signals, sleep state/stage and sleep cycle can be revealed so as to provide, e.g., the distribution of sleep respiratory events in each sleep stage, the relationship between the sleep position and the sleep stage, and the relationship between the sleep quality and the sleep disorders.

In addition, the alarm unit also can be included for performing sleep positional training and/or sleep respiratory feedback training. For example, the acquired sleep position related information can be compared with the predetermined position range for deciding an alarm behavior as the predetermined position range is met and performing the sleep position training; or the acquired sleep respiratory information can be compared with the predetermined condition for deciding an alarm behavior as the predetermined condition is met and performing the sleep respiratory feedback training; or both information can be used for providing the sleep position related training and the sleep respiratory feedback training appropriately in the same sleep duration. Further, according to different demands, the alarm unit can be, for example, mounted in the housing, mounted in another wearable device, such as a smart watch or a smart band, or mounted in an external device, such as a smartphone, without limitation.

In an embodiment, a sleep physiological system includes a housing and an adhesive wearable structure for mounting the housing on the torso of an user. The sleep physiological system also includes a control unit, mounted in the housing, at least including microcontroller/microprocessor, a communication module electrically connected to the control unit, and a power module. For acquiring sleep physiological information, a position sensor and multiple electrodes respectively electrically connected to the control unit are provided. The position sensor is used to acquire sleep position related information during sleep, and the multiple electrodes are used to acquire ECG signals and the resistance variations generated by the torso. Further, the sleep physiological system also includes an information providing interface for providing the user the information.

Particularly, since the housing is mounted on the torso, multiple electrodes can be used to acquire ECG signals and the resistance variations together. In practice, ECG signals can be acquired by two electrodes through two electrode mode, or by three electrodes through three electrode mode with the DRL electrode. The resistance variations are acquired through a loop formed by two electrodes. Therefore, depending on different demands, it can be implemented to employ two electrodes to acquire both ECG signals the resistance variations, or to employ three electrodes with sharing one electrode thereof.

Since the resistance variations are generated by the movements of chest and/or abdomen during breathing, so that through analyzing the resistance variations, it can obtain the information related to respiration, e.g., the respiratory effort for revealing if the chest and/or abdomen moves during sleep, the respiratory amplitude for revealing the degree of amplitude, and the respiratory frequency. The ECG signals can be used to reveal heart activities during sleep, e.g., heart rate, HRV and arrhythmia etc.

The sleep respiratory information described above can help the understanding of sleep apnea/hypopnea. As known, the causes of OSA and CSA are different, and the two can be distinguished through observing if the respiratory effort stops as the sleep apnea/hypopnea happens, and this is also one of the important factors for deciding the provision of sleep position training and/or sleep respiratory feedback training. For example, OSA can be treated by sleep position training and/or sleep respiratory feedback training based on different conditions, and CSA is mainly treated through the sleep respiratory feedback training.

The variations of respiratory amplitude, the variations of respiratory frequency and the variations of heart rate obtained from ECG signals also can be employed to reveal if apnea events and/or hypopnea events happened in the sleep duration. For example, when obstructive sleep apnea/hypopnea happens, the respiratory amplitude gradually decreases due to the more and more serious blockage of the upper airway and then recovers until the next event happens; the respiratory frequency rises sharply when awakeness or arousal happens and then recovers until the next event happens; and the variation of heart rate gradually slows down with the happening of sleep apnea/hypopnea and rises sharply when awakeness or arousal happens, and then recovers until the next event happens.

Accordingly, through mounting multiple electrodes, the sleep physiological system of the present disclosure can further distinguish OSA and CSA, except for the happening of sleep apnea/hypopnea. Further, through cooperating with the sleep position related information acquired by the position sensor, it can know that if the sleep apnea/hypopnea is positional. For example, through comparing the sleep respiratory events with the sleep position related information, it can obtain the distributions of sleep respiratory events corresponding to the predetermined sleep position range is met or not, so as to obtain the sleep respiratory event position correlation information, e.g., a position related apnea index, a number of position related sleep respiratory events, a duration of position related sleep respiratory events. Then, the information can be provided to the user through the information providing interface. Therefore, only one single system is mounted and only single usage is needed, and thus, the whole figure of sleep apnea/hypopnea can be revealed, which is extremely advantageous.

The information providing interface, without limitation, can be implemented to mount on the housing, e.g., LED on the housing, or to mount on an external device which communicates with the control unit via the communication module, e.g., the smart device, and LED, LCD, speaker of a computer device.

When the accelerometer is implemented as the position sensor, it can be further used to detect the vibrations of body cavity caused by snoring, which means the information of another common sleep disorder, snoring, also can be obtained, so that the relationship between the happening of snore and sleep position also can be revealed, e.g., through a position related snore index, a number of position related snorings, a duration of position related snorings. When the snoring is detected through the accelerometer, the detection result is not influenced by the environmental sounds, and even though the accelerometer is covered by cloth or quilt, e.g., as being mounted on the torso, the detection still can be performed normally. The accelerometer further can be implemented to acquire other sleep physiological information, e.g., respiratory effort for being compared with the respiratory effort acquired by another physiological sensor, and the sleep actigraph for providing information of sleep state/stage. Alternatively, it also can be implemented to additional employ an accelerometer for acquiring the information described above, without limitation.

In addition, the alarm unit also can be included, e.g., a tactile alarm unit, for performing the sleep positional training and/or the sleep respiratory feedback training. For example, the acquired sleep position related information can be compared with the predetermined position range for deciding an alarm behavior and providing alarms, e.g., vibration alarms, as the predetermined position range is met so as to perform the sleep position training; or the acquired sleep respiratory information, e.g., respiratory effort, respiratory amplitude, respiratory frequency, heart rate, snore related information etc., can be compared with the predetermined condition for deciding an alarm behavior and providing alarms, e.g., vibration alarms, as the predetermined condition is met so as to perform the sleep respiratory feedback training; or both information can be used for providing the sleep position related training and the sleep respiratory feedback training appropriately in the same sleep duration.

As to how the alarms are provided, the control unit generates a driving signal and after receiving the driving signal, the alarm unit produces at least an alarm for providing to the user, thereby achieving the purpose of sleep positional training and/or sleep respiratory feedback training, wherein the driving signal is generated according to the alarm behavior decided as described above.

In this way, except for understanding the happenings of sleep apnea/hypopnea, the training procedure(s) also can be performed in one single system, which provides comprehensive functions for the user.

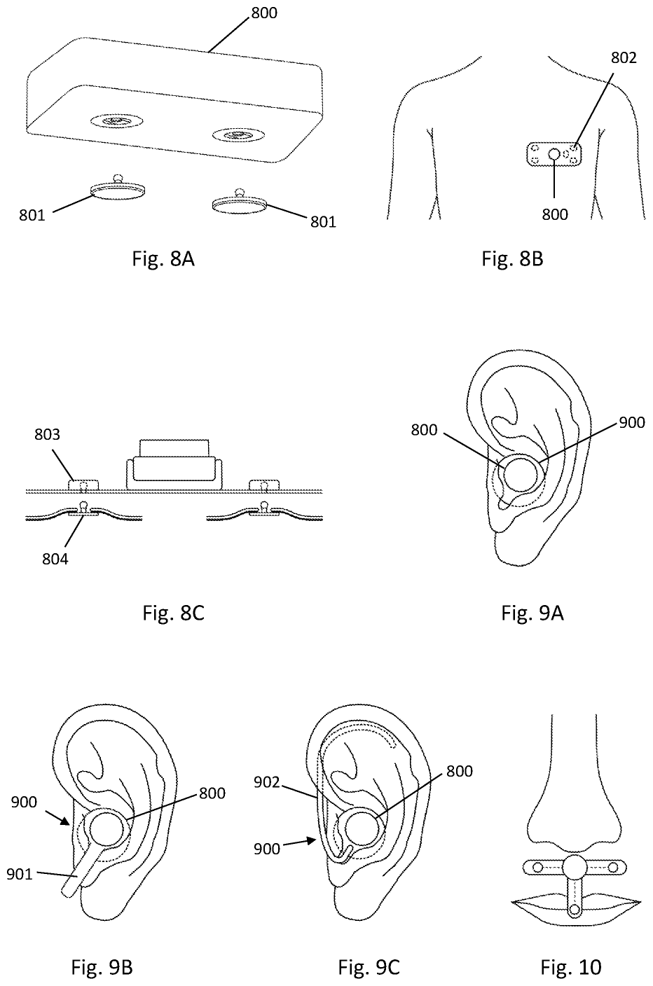

There are many types of electrodes that can be used. One advantageous option is to employ electrode patches. As known, electrode patch is a common electrode pre-formed with conductive gel, and through the conductive gel, the electrode can be adhered on the skin surface. In the present disclosure, the adhesion thereof is further used as the adhesive wearable structure for carrying the housing, namely, the electrode patch is implemented as electrode and adhesive wearable structure at the same time. In this case, as shown in FIG. 8A, it only needs to combine the housing 800 with the electrode patch 801 and then the setting is completed. For example, generally, the electrode patch adopts a structure similar to the snap button for combination, such as a male snap protrusion, so that it only needs to form a corresponding structure, such as a female snap indentation, on the housing and thus the mechanical combination between the housing and the wearable structure and the electrical connection between the electrode and the control unit both can be completed. Note that the electrode patch can be implemented to be one electrode in one patch or multiple electrodes in one patch, which can be changed depending on practical demands without limitation.

Another advantageous option is to mount the electrode(s) on a surface of the adhesive wearable structure which contacts the skin. Because the adhesive wearable structure is configured to carry the housing and also deployed on the skin surface, if the electrode(s) can be mounted on the surface of the adhesive wearable structure for contacting the skin, only one single action can complete both the settings of the electrode(s) and the housing. In practice, at least two electrodes which are electrically connected to the control unit are mounted on a bottom surface of the adhesive wearable structure. As shown in FIG. 8B, it can be implemented to be wet electrodes 802 which needs to use additional applied conductive medium, e.g., conductive gel, and in this case, the housing can be fixed through the adhesion force provided by the conductive medium or through applying adhesive material, e.g., glues, on areas other than the electrodes. Alternatively, the electrodes can be implemented to be dry electrodes which don't need to use a conductive medium, so that for ensuring the stable contact between the electrode and the skin, there are different possibilities. As shown in FIG. 8C, the wearable structure can be implemented to have at least two combining elements 803 which can be combined with at least two dry electrodes 804, for example, the combining element 803 can have an indentation structure corresponding to a protrusion structure on the dry electrode. In this case, since the dry electrode can be fixed independently, e.g., through adhesive tapes, a stable contact with the skin can be ensured without being influenced by the movement of the wearable structure.

No matter adopting the wet electrode or the dry electrode, the housing and the wearable structure both can be implemented to be removable so as to provide the possibility of changing. For example, the distance between electrodes or the distribution of electrodes can be changed through exchanging different wearable structures; or the type of electrode also can be changed, e.g., from dry electrode to wet electrode; or the electrode can be replaced by a new one, e.g., when the conductive gel of the wet electrode has lost the adhesive force.

Alternatively, the electrode and the adhesive wearable structure also can be implemented to be independent of each other, e.g., the adhesive wearable structure is used to carry the housing, and the electrodes are extended from the housing and then fixed.

In an embodiment, a system physiological system includes a housing and an ear plug type wearable structure for mounting the housing on an ear. The sleep physiological system also includes a control unit, mounted in the housing, at least including microcontroller/microprocessor, a communication module electrically connected to the control unit, and a power module. Further, the sleep physiological system includes at least a physiological sensor electrically connected to the control unit for acquiring at least a sleep physiological information of the user during sleep, and an audible alarm unit electrically connected to the control unit for providing at least an audio alarm.

Due to the adoption of the ear plug type wearable structure which has the ear as the main installing position, it is suitable to employ audio alarms, thereby simplifying the installation procedure. In practice, a sounding element can be used for generating sounds, e.g., a speaker or a buzzer.

The at least a sleep physiological information can be implemented to include the sleep position related information and/or the sleep respiratory information, so that the at least a physiological sensor can be different accordingly. For example, the optical can be employed to mount on the ear for acquiring sleep respiratory information, such as heart rate and/or blood oxygen saturation; the accelerometer can be employed to mount on the ear to acquire various sleep physiological information, such as sleep position related information, snore related information and/or heart rate; and the microphone also can be employed to mount on the ear for acquiring sleep respiratory information, such as snore related information and/or the variation of breathing sounds. Further, it also can be implemented to employ more than two physiological sensors, for example, the accelerometer for acquiring sleep position related information and the snore related information is employed together with the optical sensor for acquiring heart rate and/or blood oxygen saturation.

According to the information described above, for example, it is able to know that, in the sleep duration, the sleep position of the user is supine or non-supine and also if the sleep respiratory events happened, such as blood physiological related sleep respiratory events and snore events. All these are the bases for performing the sleep position training and the sleep respiratory feedback training. Therefore, by cooperating with the audio alarms, when the sleep position related information meets the predetermined position range and/or when the sleep respiratory information meets the predetermined condition, it is able to perform the sleep position training, the sleep respiratory feedback training or both in the same sleep duration. Accordingly, one single system mounted on the ear can provide multiple functions including, bot not limit, the detection of sleep position, the evaluation of the happenings of sleep disorders, and the provision of the sleep position training and/or the sleep respiratory feedback training, which achieves a simple but powerful system.

As to how the alarms are provided, identically, the control unit generates a driving signal and after receiving the driving signal, the audible alarm unit produces at least an audio alarm for providing to the user, thereby achieving the purpose of sleep positional training and/or sleep respiratory feedback training, wherein the driving signal is generated at least according to an audio alarm behavior which is decided as the at least a sleep physiological information and/or the sleep position related information respectively met the predetermined position range and/or the predetermined condition.

In an embodiment, a sleep physiological system includes a housing, at least a wearable structure, a control unit at least including microcontroller/microprocessor, at least an airflow sensor electrically connected to the control unit, a physiological sensor electrically connected to the control unit, a communication module electrically connected to the control unit, and a power module. Through the at least a wearable structure, as shown in FIG. 10, the housing 800 and the least an airflow sensor 1001 are mounted between the nose and the mouth of a user for acquiring the breathing flow variations of the user during sleep. The physiological sensor is used to acquire another kind of sleep physiological information. The at least an airflow sensor can be implemented as thermistors, thermocouples, or the nasal cannula/pressure transducer. The nasal cannula/pressure transducer detects the flow variations of breathing flows, and the thermistors and thermocouples detect the temperature variations caused by breathing flows which can be selected to set at two locations (near two nostrils) or three locations (near two nostrils and the mouth).

As known, the most direct way to understand breathing is to detect the breathing flow, and thus, the apnea events and/or hypopnea events can be derived therefrom. Therefore, when the size of the housing is small enough, e.g., smaller than mm, it is possible to place the housing with the airflow sensor between the nose and the moth, as shown in FIG. 10, through a suitable wearable structure. For example, the wearable structure can be implemented as at least an adhesive element for fixing the housing which can be selected to adhering the area between the nose and the mouth or the areas aside the moth; or the wearable structure also can be implemented as a fixing structure for clamping the nasal septum or the alae of the nose; or the wearable structure can be implemented to utilize both adhering and clamping capabilities for fixing; or the wearable structure can be implemented to be any other structure capable of achieving the fixing. In this case, the material of the housing, except for the plastic which is commonly used, also can be selected to adopt a soft or flexible material for better comfort.

The physiological sensor can be used to acquire more sleep physiological information during sleep, for example, it can be implemented as the accelerometer for acquiring sleep position related information and snore related information, or as the optical sensor for acquiring blood oxygen saturation and heart rate, or as the microphone for acquiring the snore related information. No matter which kind of information is acquired, through cooperating with the breathing flow variation, a meaningful combination for revealing sleep disorder can be obtained.

The at least two wearable structures can be implemented to be two wearable structures, which respectively are capable of being removed from the housing, for mounting the housing on different body portions, e.g., the forehead, the ear, the torso, the finger, the wrist, the arm etc. Accordingly, the physiological sensor can be configured to acquire various kinds of physiological information, e.g., blood oxygen saturation, heart rate, snore related information, sleep position, sleep actigraph, and daytime actigraph, for providing another usage option. Particularly, since the mounting of the airflow sensor is limited to locate between the nose and the mouth, the wearable structure thereof is preferably implemented to be removable from the housing, so that when the housing is combined with another wearable structure for mounting on another body portion, the whole structure can be simplified.

Furthermore, for hygiene and/or for the use by multiple people, even the housing is not changed to another body portion, it is also preferably that the airflow sensor and the housing are implemented to be removable, so that the airflow sensor can be exchanged.

The sleep physiological system can further include a wearable device. The wearable device has another physiological sensor, e.g., optical sensor and accelerometer, for mounting on, e.g., the wrist, the finger, the torso, the head etc., so as to acquire additional sleep physiological information, such as blood oxygen saturation, heart rate, respiratory effort, snore related information, sleep position, and sleep actigraph. Thereby, the comparison can be performed among more sleep physiological information. For example, in the case of the airflow sensor already can acquire the breathing flow variation, through cooperating with the respiratory effort acquired by the accelerometer, it can decide that the apnea events and/or the hypopnea events are related to OSA in which the chest and the abdomen still move during events or CSA in which the chest and the abdomen are not moving during events.

In addition, the sleep physiological system can further include the alarm unit for providing alarms according to the breathing flow variations and/or the sleep physiological information. If the sleep physiological information includes sleep position, then it is able to perform the sleep positional training; and/or the breathing flow variation and/or other sleep physiological information can be used for performing the sleep respiratory feedback training. Further, according to different demands, the alarm unit can be mounted in the housing, or can be implemented by an external device communicated with the communication module in the housing, such as a smartphone, a smart watch and a smart band.

So far, for the sleep physiological system of the present disclosure, how to be deployed on the user's body is important, especially the performing of tactile alarms, such as vibration alarms, needs the housing to contact with the skin in a stable and close way, thereby the vibrations can be transmitted to the user effectively. Further, the acquisitions of physiological information of many kinds of physiological sensors also depend on the contact thereof with the skin, for example, the best condition for the optical sensor to acquire is to slightly press the skin, and the position sensor and the accelerometer should be closely mounted on the skin to detect the sleep position and the cavity vibration caused by snoring, the movements of chest and abdomen and the sleep actigraph.

One option is to adhere the housing on the skin, e.g., through an adhering structure, only if the size of the housing is suitable. Another option is to employ the elastic clothing to be the medium for mounting the housing so as to place the housing closely on the skin.

The implementation is to provide a fixing structure for producing a fixing force to mount the housing on the clothing, and at least a portion of the clothing provides an elastic force for applying force to the skin surface when the user wears the clothing, such that a close stacked structure including the housing, the clothing and the skin surface can be formed, and through this closely stacked structure and the elastic force, the housing can be closely attached to the body surface, thereby no matter the provision of tactile alarms or the mounting of the physiological sensor, both can be achieved effectively.

The housing can be selected to locate at different positions. For example, the housing can be located between the clothing and the skin, or be located outside the clothing and stay close to the body surface. If the signal acquisition of the physiological sensor needs to contact the skin, e.g., the optical sensor, the surface of the physiological sensor should be placed to contact the skin.

The fixing manner of the fixing structure on the clothing can be different. For example, it can be implemented to adhere to the clothing, e.g., by utilizing the adhering structure to adhere the housing on the clothing, or it also can be implemented to be a clamp structure, e.g., a clamp structure using mechanical force or magnetic force.

In a preferable embodiment, the clamp structure can have an accommodating space for combining with the housing, and then, after the clamp structure is clamped on the clothing, the installation of the housing is conveniently completed. The accommodating space can be implemented to be at the inside or the outside of the clothing. If the physiological sensor is located on the surface of the housing, when the housing is in the accommodating space, it should pay attention to expose the physiological sensor.

When adopting the magnetic clamp structure, it is preferable that one magnetic object is mounted with the housing for attracting the other magnetic object through the clothing. The magnetic object can be mounted in the housing, e.g., through being added in the housing, or can be implemented to directly utilize the battery, which is made of metal and capable of being attracted by the other magnetic object, in the housing, or can be located outside the housing, e.g., through being placed in the accommodating space with the housing or being embedded in the accommodating space. Further, it is preferable to have a flexible connecting element connected between the accommodating space and the other magnetic object, so that the clamping can be achieved through the bending property thereof.

Note that the elastic force of the clothing can come from the material of the clothing, e.g., the elastic cloth, or an elastic element added on the clothing, e.g., an elastic band sewn on the clothing. In addition, the clothing not only can be the clothes, e.g., the tight fitting clothes, underwear, and pants, also can be other clothing surrounding the body, e.g., a surrounding belt, such as the RIP sensor surrounding the torso. There are many options without limitation.

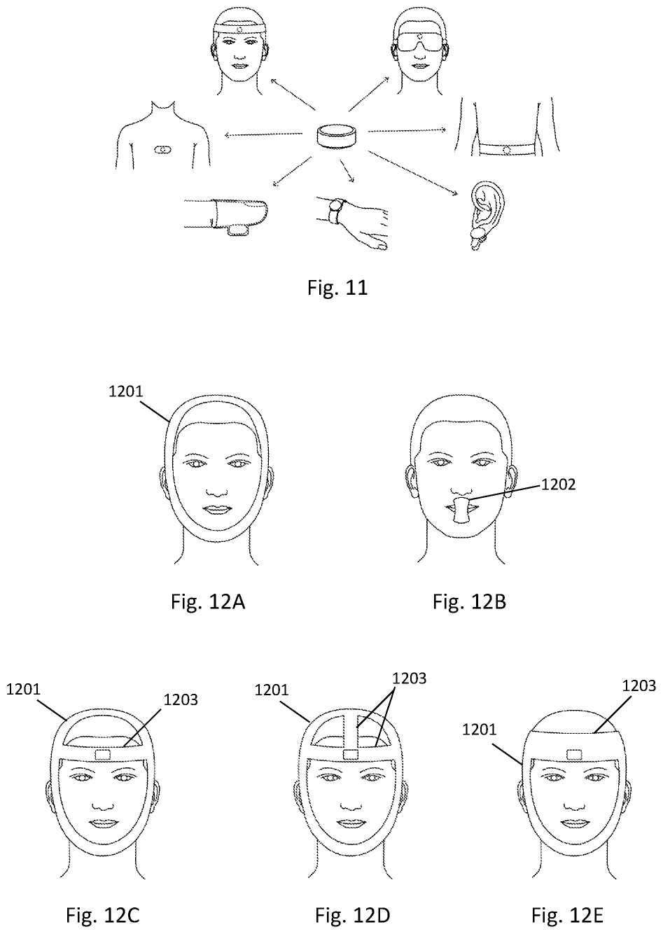

Accordingly, the sleep physiological system of the present disclosure can be implemented differently according to different demands and hardware configurations, e.g., to use the dispersed framework, or to change the mounting position, thereby as shown in FIG. 11, through the housing being implemented to be removable from the wearable structure for cooperating with different wearable structures, the system can conveniently and easily satisfy the demands for mounting at different body portions.

In another aspect of the present disclosure, an oral closing auxiliary is further provided for helping to improve OSA symptom in addition to utilizing the alarms to provide the sleep position training and/or the sleep respiratory feedback training. The oral closing auxiliary is used to mount near the upper airway for improving the collapse of upper airway.

A chin belt 1201, as shown in FIG. 12A, is a known oral closing auxiliary for improving snoring and/or OSA symptom. When the chin belt is surrounding the head, the belt will apply a force on the chin for forcing the chin to move upward which affects the muscle around the throat, and thus, even the muscle is relaxed during sleep, the upper airway still can remain unobstructed through the oral closing auxiliary keeping the mouth closed, thereby improving snoring and/or OSA symptom.

Another known oral closing auxiliary for improving the obstruction and/or collapse of upper airway is an adhesive oral positioning element 1202, shown in FIG. 12B. The adhesive oral positioning element which has the similar function with the chin belt forces the upper and lower lips in a closed state, so as to prevent the mouth from opening during sleep so as to affect the muscle of the throat and keep the upper airway unobstructed. Another function of the adhesive oral position element is to avoid breathing through the mouth.

Because the effects of improving snoring and/or OSA symptom achieved by mounting the oral closing auxiliary vary from person to person, e.g., everyone has a different throat structure and different sleep position which may influence the opening degree of the upper airway, if the physiological signals can be acquired at the same time, such as blood oxygen saturation, heart rate, variations of breathing flows, respiratory effort, which can reveal if the obstruction of upper airway is solved, e.g., if the happening number of ODI events, low oxygen level events, heart rate variation sleep respiratory events, snore events, apnea events and/or hypopnea events is reduced. This is an effective combination.

Therefore, the oral closing auxiliary can cooperate with many kinds of physiological sensors, e.g., the optical sensor, the accelerometer, the airflow sensor, the piezoelectric vibration sensor, the piezoelectric motion sensor, electrodes for detecting body resistance, the RIP sensor, and/or the microphone. For example, at first, the user can utilize the physiological sensor alone to monitor the physical condition, e.g., utilize the optical sensor to know if there are blood physiological related sleep respiratory events happened, or utilize the accelerometer, the microphone and/or the piezoelectric vibration sensor to acquire the snore related information so as to know if there are snore events happened, or utilize other physiological sensor to acquire other sleep respiratory events. Then, the oral closing auxiliary can be used to keep the upper airway unobstructed together with physiological sensor which provides the user how is the effect thereof, e.g., if the number of events is reduced. Further, the physiological information also can be used as the base to adjust the mounting of the oral closing auxiliary, e.g., the tightness and the angle of the chin belt, and the stickiness and the coverage of the adhesive oral positioning element.

In an embodiment, the oral closing auxiliary can cooperate with a sleep physiological device which includes a control unit at least including microcontroller/microprocessor, a physiological sensor electrically connected to the control unit for acquiring a sleep respiratory information of a user during sleep, a communication module electrically connected to the control unit, a power module, and a wearable structure for mounting the device on the user's body. The control unit is configured to analyze the sleep respiratory information for obtaining sleep respiratory events, which are provided to the user through the information providing interface, and thus, the user can know the effect achieved by using the oral closing auxiliary. All the physiological sensors described above can be used to cooperate with various wearable structures, e.g., finer-worn structure, wrist-worn structure, head-mount structure, belt, patch, for mounting on various body portions, e.g., the finger, the wrist, the torso, the forehead, the ear, the area between nose and mouth, without limitation. Particularly, the device/physiological sensor also can be directly mounted on the oral auxiliary.

Further, the position sensor also can be included to acquire sleep position related information. In this case, through comparing the sleep respiratory information with the sleep position related information, it can reveal if the sleep disorder is positional which helps to classify the type thereof.

In addition, the alarm unit also can be included, so that when the sleep respiratory events are happening, the alarms can be provided to the user for performing sleep respiratory feedback training. The cooperation of the training and the oral closing auxiliary makes the effects doubled. Further, if the physiological sensor and the position sensor are both included, the training can be the sleep position training and/or the sleep respiratory feedback training.

The oral closing auxiliary also can be implemented to cooperate with the position sensor and the alarm unit. For example, in an embodiment, a sleep physiological device includes a control unit at least including microcontroller/microprocessor, a position sensor electrically connected to the control unit for acquiring a sleep position related information of a user during sleep, an alarm unit electrically connected to the control unit for providing at least an alarm to the user during sleep, a communication module electrically connected to the control unit, a power module, and a wearable structure for mounting the device on the user's body so as to perform a sleep position training. In this case, since the upper airway can be kept unobstructed through the help of the oral closing auxiliary, the effect of sleep position training can be improved significantly. Further, through the information providing interface, the user can understand how the usage of the oral closing auxiliary influences the sleep position and the alarm behavior. In another embodiment, a physiological sensor can be further included, e.g., the optical sensor, the accelerometer, the airflow sensor, the piezoelectric vibration sensor, the piezoelectric motion sensor, electrodes for detecting body resistance, the RIP sensor, and the microphone etc., for acquiring sleep respiratory information to obtain the sleep respiratory events, which can be provided through the information providing interface to let the user know the effect of the oral closing auxiliary on the improvement of the sleep disorder.

The procedures of the sleep position training and/or the sleep respiratory feedback training are described below. The acquired sleep position related information is compared with the predetermined position range for deciding an alarm behavior and providing alarms as the predetermined position range is met so as to perform the sleep position training. The acquired sleep respiratory information, e.g., snore related information, blood oxygen saturation, respiratory effort, heart rate etc., is compared with the predetermined condition for deciding an alarm behavior and providing alarms as the predetermined condition is met so as to perform the sleep respiratory feedback training. As to how the alarms are provided, the control unit is configured to generate a driving signal and after receiving the driving signal, the alarm unit produces at least an alarm for providing to the user, thereby achieving the purpose of sleep positional training and/or sleep respiratory feedback training, wherein the driving signal is generated according to the alarm behavior decided as described above.

The physiological sensor, the sleep position sensor and/or the alarm unit can be implemented, e.g., to be any suitable sleep physiological device, sleep respiratory device or sleep alarm device described above, or to be in another wearable device or an external device. Further, if the position of the oral closing auxiliary is suitable, it also can be used to mount the physiological sensor, the sleep position sensor and/or the alarm unit, e.g., to locate the airflow sensor between the nose and the mouth, to mount the sleep position sensor/the accelerometer/the microphone on the top of the head or the chin, which makes the arrangement simpler.

Particularly, when adopting the head-mount structure, especially as the belt type, it can be further implemented to combine the head-mount structure with the chin belt, so as to enhance the stability.

The general chin belt, as shown in FIG. 12A, may slide due to covering the hair to cause an unstable installation, so that during sleep, the chin belt may fall off and thus the effect may be influenced. As shown in FIG. 12C, when the chin belt is combined with the head-mount structure 1203, which is mounted on the forehead and is crossed connected with the chin belt 1201, the combination therebetween provides the positioning forces in two direction, namely the vertical and horizontal directions, so that the mutual interference between two belts can effectively reduce the sliding and make sure a stable installation.

Other variations are also possible. For example, as shown in FIG. 12D, one more belt can be mounted on the top of the head. Or as shown in FIG. 12E, based on the horizontal head mount belt which can provide a stable interference with the head, the chin belt may only surround the lower portion of the head without sliding, and in this case, the head mount structure can be further varied, e.g., to cover the most portion of the top of the head or to be a hat-like structure, without limitation.

The combination manner between the chin belt and the head-mount structure can be implemented differently, e.g., to utilize the Velcro, a buckling structure, or a mutually penetrating structure, and also, the combination can be implemented to be removable or sewn together.

Noted that, in all the embodiments described above, no matter the analysis of the physiological information, the judgment of sleep respiratory events, the decision of alarm providing and/or the decision of alarm behavior, all are achieved by various programs, and without limitation, the program(s) can be implemented to preload in any of the wearable device and/or the external device for performing calculation so as to achieve a most convenient operation procedure for the user.

In the embodiments described above, the wearable structure for mounting the position sensor, the housing, the device and/or the system on the user' body can be varied according to the mounting location, e.g., the material thereof can be different, and if appropriate, one wearable structure can be used to mount at different body portions. For example, the belt type wearable structure can be mounted on any body portion that can be surrounded, e.g., the head, the neck, the chest, the abdomen, the arm, the wrist, the finger, the leg etc. The material thereof can be varied, e.g., to be the fabric, the silicone, the rubber etc. Further, the adhesive structure, such as the patch, can almost be mounted on any portion of the body. In addition, the particular body portion can have exclusive wearable structure, for example, the eye mask can be used to mount on the head which is especially suitable for sleep, and an arm-worn structure, a wrist-worn structure and a finger-worn structure can respectively be used to mount on the arm, the wrist and the finger. Therefore, the implementation of the wearable structure can be varied depending on the different demands without being limited by the embodiments described above.

When the wearable structure is used to carry the housing/device, the combination therebetween are also variable. For example, the combination can be achieved by adhering, clamping through mechanical or magnetic force, sleeving through forming a sleeve on the wearable structure, stuffing through forming a space for stuffing the housing/device, and/or any other suitable manner, without limitation.

In the embodiments described above, any information that is acquired by the physiological sensor or obtained after calculation, or is related to the operation procedure, is provided through the information providing interface to the user, and the information providing interface can be implemented to mount on any one or more devices of the system.

In the embodiments described above, the acquisitions of various sleep physiological information can be implemented to utilize any kind of physiological sensor, to be at any body portion, and to perform any calculation according to the acquired physiological information, and the duplicated contents are omitted for simplicity. The claimed range of the present disclosure is not limited thereby.

Furthermore, the devices in the embodiments can adopt the circuitry mentioned above and can be varied according to the type of physiological information to be acquired and the mounting position, and the duplicated contents are also omitted for simplicity. The claimed range of the present disclosure is not limited thereby.

In addition, the embodiments described above can be performed alone or combined in part or as a whole without escaping the claimed range of the present disclosure.

The example embodiments of the disclosure described herein do not limit the scope of the invention, since these embodiments are merely examples of the embodiments of the invention, which is defined by the appended claims and their legal equivalents. Any equivalent embodiments are intended to be within the scope of this invention. Indeed, various modifications of the disclosure, in addition to those shown and described herein, such as alternative useful combinations of the elements described, may become apparent to those skilled in the art from the description. Such modifications and embodiments are also intended to fall within the scope of the appended claims.

In the present disclosure, where conditions and/or structures are not specified, the skilled artisan in the art can readily provide such conditions and/or structures in view of the present disclosure, as a matter of routine experimentation.

What is claimed is:

1. A sleep alarm method, comprising steps of:
providing a sleep physiological system at least comprising a control unit, at least a physiological sensor, a position sensor, an alarm unit and a wearable structure;
the at least a physiological sensor acquiring at least a sleep respiratory information of a user in a sleep duration;
the position sensor acquiring a sleep position related information of the user in the sleep duration;
providing a sleep respiratory information analysis program for comparing the at least a sleep respiratory information with a predetermined condition so as to decide at least a sleep respiratory event of the user;
providing a sleep position analysis program for comparing the sleep position related information with a predetermined position range;
when the sleep position related information meets the predetermined position range, providing a first set of alarming conditions, and when the sleep position related information is out of the predetermined position range, providing a second set of alarming conditions, wherein at least one of the first set of alarming conditions and the second set of alarming conditions comprises a sleep respiratory event criterion;
providing an alarm deciding program for deciding at least an alarm behavior according to a corresponding set of alarming conditions based on a comparison result between the sleep position related information and the predetermined position range;
the control unit producing a driving signal according to the at least an alarm behavior; and
the alarm unit producing at least an alarm after receiving the driving signal for influencing a sleep position and/or a sleep respiratory state of the user.

2. The method as claimed in claim 1, wherein the physiological sensor comprises at least one selected from a group consisting of: an optical sensor, an accelerometer, a microphone, an airflow sensor, a piezoelectric motion sensor, a piezoelectric vibration sensor, a RIP sensor and electrodes for detecting body resistance.

3. The method as claimed in claim 2, wherein the at least a sleep respiratory event comprises at least one selected from a group consisting of: an ODI event, a low oxygen level event, a heart rate variation sleep respiratory event, a snore event, an apnea event and a hypopnea event.

4. The method as claimed in claim 2, wherein the accelerometer is implemented to be the physiological sensor and the position sensor at the same time.

5. The method as claimed in claim 1, wherein the first set of alarming conditions and the second set of alarming conditions respectively at least comprise a time range criterion.

6. The method as claimed in claim 5, wherein the time range criterion is implemented as being based on an absolute time, on a delay time or on a specific physiological condition.

7. The method as claimed in claim 5, wherein when the sleep position related information meets the predetermined position range, the first set of alarming conditions comprises at least one of the time range criterion and the sleep respiratory event criterion.

8. The method as claimed in claim 5, wherein when the sleep position related information is out of the predetermined position range, the second set of alarming conditions comprises the time range criterion and the sleep respiratory event criterion.

9. A system physiological system, comprising:
a housing;
a control unit, at least comprising microcontroller/microprocessor;
a position sensor, electrically connected to the control unit for acquiring a sleep position related information of a user in a sleep duration;
a power module; and
a wearable structure, for mounting the housing on the user's body,
wherein the system further comprises an alarm unit for producing at least an alarm to provide to the user; and
the system further comprises a physiological sensor for acquiring at least a sleep physiological information of the user in the sleep duration, and
wherein the system compares the at least a sleep physiological information with a predetermined condition to decide if the user meets a predetermined sleep respiratory condition, and when the user meets the predetermined sleep respiratory condition, the system enters an alarm producing state; and
in the alarm producing state, the control unit is configured to generate a driving signal, and after receiving the driving signal, the alarm unit produces the at least an alarm for providing to the user, wherein the driving signal is generated according to an alarm behavior, which is decided when the sleep position related information meets a predetermined position range through comparing with the predetermined position range.

10. The system as claimed in claim 9, wherein the physiological sensor comprises at least one selected from a group consisting of: an optical sensor, an accelerometer, a microphone, an airflow sensor, a piezoelectric motion sensor, a piezoelectric vibration sensor, a RIP sensor and electrodes for detecting body resistance.

11. The system as claimed in claim 10, wherein the accelerometer is implemented to be the physiological sensor and the position sensor at the same time.

12. The system as claimed in claim 9, wherein the physiological sensor is mounted in the sleep physiological device and electrically connected to the control unit.

13. The system as claimed in claim 9, wherein the system further comprises another wearable device, and at least one of the physiological sensor and the alarm unit is mounted in the another wearable device.

14. The system as claimed in claim 9, wherein the system further comprises an external device and the physiological sensor is implemented to mount in the external device.

15. The system as claimed in claim 9, wherein the system further comprises a wireless communication module electrically connected to the control unit for performing a wireless communication.

16. The system as claimed in claim 9, wherein the predetermined sleep respiratory condition comprises at least one selected from a group consisting of: an ODI event, an low oxygen level event, a heart rate variation sleep respiratory event, a snore event, an apnea event and a hypopnea event.
